(19) United States
(12) Patent Application Publication
(10) Pub. No.: US 2010/0077654 A1
(43) Pub. Date: Apr. 1, 2010

(54) SYSTEMS AND METHODS FOR PRODUCING BIOFUELS FROM ALGAE

(75) Inventors: Benjamin Chiau-pin Wu, San Ramon, CA (US); David Stephen, Davis, CA (US); Gaye Elizabeth Morgenthaler, Woodside, CA (US); David Vancott Jones, Woodside, CA (US)

Correspondence Address: JONES DAY, 222 EAST 41ST ST, NEW YORK, NY, 10017, US

(73) Assignee: LiveFuels, Inc., San Carlos, CA (US)

(21) Appl. No.: 12/565,610

(22) Filed: Sep. 23, 2009

Related U.S. Application Data
(60) Provisional application No. 61/099,502, filed on Sep. 23, 2008.

(51) Int. Cl. C10L 1/188; C12P 7/64; C12M 1/00

(52) U.S. Cl. 44/385; 435/134; 435/289.1

(57) ABSTRACT

The invention provides systems and methods for producing biofuel from algae wherein the algae and fishes are co-cultured in a body of water. The methods further comprise inducing the algae to accumulate lipids by environmental stress, and concentrating the algae prior to extraction of the algal oil. The systems of the invention comprise at least one growth enclosure, means for concentrating algae, and means for subjecting algae to environmental stress.

The application claims the benefit of U.S. Provisional Patent Application Ser. No. 61/099,502, filed Sep. 23, 2008, which is incorporated by reference herein in its entirety.

### 1. INTRODUCTION

The invention relates to systems and methods for producing biofuels from algae.

### 2. BACKGROUND OF THE INVENTION

The United States presently consumes about 42 billion gallons per year of diesel for transportation. In 2007, a nascent biodiesel industry produced 250 million gallons of a bio-derived diesel substitute produced from mostly soybean oil in the U.S. Biodiesel are fatty acid methyl esters (FAME) made typically by the base-catalyzed transesterification of triglycerides, such as vegetable oil and animal fats. Although similar to petroleum diesel in many physicochemical properties, biodiesel is chemically different and can be used alone (B100) or may be blended with petrodiesel at various concentrations in most modern diesel engines. However, a practical and affordable feedstock for use in biodiesel has yet to be developed that would allow significant displacement of petrodiesel. For example, the price of soybean oil has risen significantly in response to the added demand from the biodiesel industry, thus limiting the growth of the biodiesel industry to less than 1% of the diesel demand.

It has been proposed to use algae as a feedstock for producing biofuel, such as biodiesel. Some algae strains can produce up to 50% of their dried body weight in triglyceride oils. Algae do not need arable land, and can be grown with impaired water, neither of which competes with terrestrial food crops. Moreover, the oil production per acre can be nearly 40 times that of a terrestrial crop, such as soybeans. Although the development of algae presents a feasible option for biofuel production, there is a need to reduce the cost of operating an algae culture facility and producing the biofuel from algae. The fall in oil price in late 2008 places an even greater pressure on the fledgling biofuel industry to develop inexpensive and efficient processes. The present invention provides a cost-effective and energy-efficient approach for growing algae and converting algae into biofuel.

### 3. SUMMARY OF THE INVENTION

The invention provides systems and methods for producing biofuel from algae that are cost-effective and energy efficient. In one embodiment, the methods involve culturing algae and a plurality of fish in a common body of water, wherein the conditions of the body of water that affect algal growth are favorably modified by the plurality of fish to promote growth of the algae. The methods also involve inducing the algae to accumulate lipids by a stressor, harvesting the algae from the culture, extracting the lipids from the algae, and converting the lipids into a biofuel feedstock or a biofuel. The invention also encompasses methods for making a liquid fuel comprising processing a biofuel feedstock of the invention. Non-limiting examples of liquid fuels that can comprise biofuels made by the methods of the invention include but are not limited to diesel, biodiesel, kerosene, jet-fuel, gasoline, JP-1, JP-4, JP-5, JP-6, JP-7, JP-8, JPTS, Fischer-Tropsch liquids, alcohol-based fuels, including an ethanol-containing transportation fuel or cellulosic biomass-based fuel, or algae pyrolysis oil-derived fuels.

The conditions of the water modified by the fish comprise but are not limited to nitrogen concentration, phosphorous concentration, carbon dioxide level, oxygen level, zooplankton population, mollusk population, crustacean population, and temperature uniformity. Such conditions in the water can be controlled by the systems of the invention. Applicable methods for controlling aquatic conditions in an enclosure or a zone within an enclosure include confining a plurality of fish, changing the total number of fish or the number of fish of any one or more species, and adjusting the degree of mixing. The method can further comprise measuring the content of lipids in a sample of the algae and repeating the growing step and inducing step at least one time after the measuring step. The method can further comprise concentrating the algae to form an algal composition prior to the inducing step, the harvesting step, or both the inducing step and the harvesting step. One of the stressor that can be used to induce synthesis and/or accumulation of lipids is culturing the algae at a concentration where one or more nutrients are limiting.

In various embodiments, the algae grown by methods of the invention comprise freshwater species, marine species, briny species of microalgae or species of microalgae that live in brackish water. The algae composition can comprise at least one species of cyanobacteria, Isochrysis, Amphiprora, Chaetoceros, Scenedesmus, Chlorella, Dunaliella, Spirulena, Coelastrum, Micractinium, Euglena, or Dunaliella. The fishes used in the invention can be herbivores, zooplanktivores, detritivores, piscivores, carnivores, or a combination of any two or more of the foregoing trophic types of fishes, and can include any freshwater species, marine species, briny species, or species that live in brackish water. Preferably, the fishes are not obligate phytoplankton feeders. In certain embodiments, the body of water in which the algae and fishes are cultured is supplemented with carbon dioxide.

In another embodiment, the systems of the invention for culturing algae comprises a growth enclosure comprising an aquatic composition or a body of water in which the algae and fishes are cultured wherein the water conditions are favorably and controllably modified by the fishes. The system optionally comprises means for controlling the aquatic conditions of an enclosure, an induction enclosure wherein the algae is induced to accumulate lipids by a stressor, a means for concentrating the algae, a means for measuring the content of lipids in the algae, a means for harvesting the algae, a means for extracting the lipids from the algae. In one embodiment, the means for concentrating algae is a foam fractionation unit as shown in the figures.

### 4. BRIEF DESCRIPTION OF DRAWINGS

FIG. 1 provides an overview of a system 100 for growing algae for biofuel production. The exemplary system is a pond 101 inoculated with selected algal species 201 and comprises zooplankton feeding fishes 202 and detritus feeding fishes 203. The pond comprises a cage 301 in which high value fishes 204 are kept. The pond further comprises a number of foam fractionation units 302 which are serially connected such that the outlet of the first unit is directed to the inlet of the second unit, and so on. The concentrated algae composition 205 is connected to an induction chamber 304 placed inside the pond or an induction chamber 305 placed outside the pond. Optionally, a concentration device 303 is installed to concentrate and convey the concentrated algae to the induction chamber. After the algae has been subjected to stress in the induction chamber, they are conveyed to a unit for harvesting and dewatering 306. The dewatered algae composition 206 is then transported to a biofuel processing facility 307.

FIG. 2 shows the side view of an exemplary algae concentration system using a series of foam fractionation units with vertical water circulation. The floating drums 310 with open bottoms are placed inside a pond 101 having a water column 102. Water enters the drum from the bottom 311 and exits at the top 312. A compressed air line 313 supplies air through diffusers 314 inside the drums to generate bubbles. Foam fractions 315 formed at the top are conveyed to the next drum below the water line. The foam fractions from the last unit is conveyed via a connecting means 316 to an induction chamber 304, 305 or a dewatering/harvesting unit 306.

FIG. 3 shows an alternative arrangement of the foam fractionation devices described in FIG. 2. The pond 101, shown in plan view, comprises two chambers: a first chamber 103 and a second chamber 104. The chambers with closed bottoms can be made of plastic. The system further comprise a pump 105 for pumping out the condensed foam fractions. In the pond but outside the first chamber are a number of floating drums 310 as depicted in FIG. 2. The floating drums in the pond are fluidically connected in parallel to the first chamber 103 such that foam fractions 315 generated in the pool are conveyed into the first chamber. The floating drums in the first chamber in turn generate foam fractions from the concentrated algae composition. The drums are fluidically connected in parallel to the second chamber 104. The foam fractions collected in the second chamber are pumped via a connecting means 316 to an induction chamber 304, 305 or a dewatering/harvesting unit 306.

FIG. 4A (isometric view) and FIG. 5 show a foam fractionation unit which can be configured linearly, spirally (FIG. 4C in plan view) or concentrically (FIG. 4B in plan view) within a pond 101. The unit comprises a plurality of barriers 400, 402 which float above the bottom of the pond 106 and can be made of plastic. The barriers are made buoyant near the top of the pond surface 107 by pipe floats 401. Gas diffusers 314 are placed at the bottom of the water columns 410 that are trapped between the barriers. Bubbles are generated by the diffusers and rise to the top of the water column. The barriers 400, 402 have slightly different heights and are shaped such that foam fractions 315 that rise to the top spills over into a predetermined neighboring water column. For example, barrier 400 can be slightly higher than barrier 402. In a spiral or concentric configuration, the barriers are arranged so that the foam fractions spill onto a neighboring water column towards the center. The foam fractions 315 spill over successive barriers towards the center where they condense in a container 404 with a pump 105 and is pumped out via a connecting means 316 to an induction chamber 304, 305 or a dewatering/harvesting unit 306. In a linear configuration, the barriers are arranged so that the foam fractions spill into neighboring water columns in the same direction towards one end of the pond where the foam condenses and is collected and pumped out. A foam breaker 403 can be used to help condense the foam.

FIG. 6 shows a conical foam fractionation unit 420 positioned in a pond 101 wherein the sloped top produces foam 315 better than a drum or barrel shape device. The unit floats above the bottom of the pond 106 with the water level 107 near the top of the unit. The bubble forming devices 421 are arranged radially around the bottom of the device. Water exits from outlet 422. The diameter of the base of the conical unit is 8 feet.

FIG. 7 shows an inclined foam fractionation device with vertical water circulation. The device is made with a 15 inches polyvinylchloride pipe 500, placed with one end on the bottom of the pond 106 and inclining at an angle in a 4 feet water column 501. Bubbles are formed within the device with micro-pore air diffuser 502 connected to a high pressure compressed air source 503 and travel upwards. Foam forms at the top of the pipe and travels towards the collection point 504. Water exits the device at 505 near the surface of the pond 107. A baffle control 506 is provided inside the device to regulate flow and separate the foam from the water.

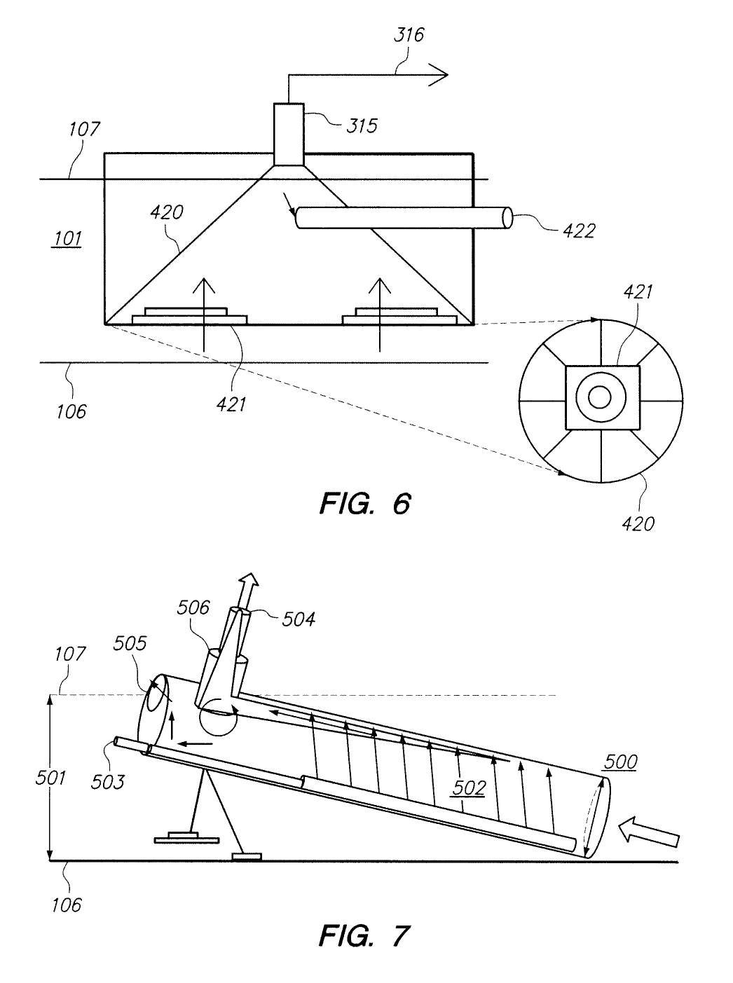

FIG. 8 is a map of an inland fish farm located in southern California with 17 river-fed, algae-containing fish ponds.

FIG. 9 shows the relative amounts of C12 to C22 saturated and unsaturated fatty acids in the algal oil extracted by ether after acid hydrolysis. For comparison, palm oil is also analyzed.

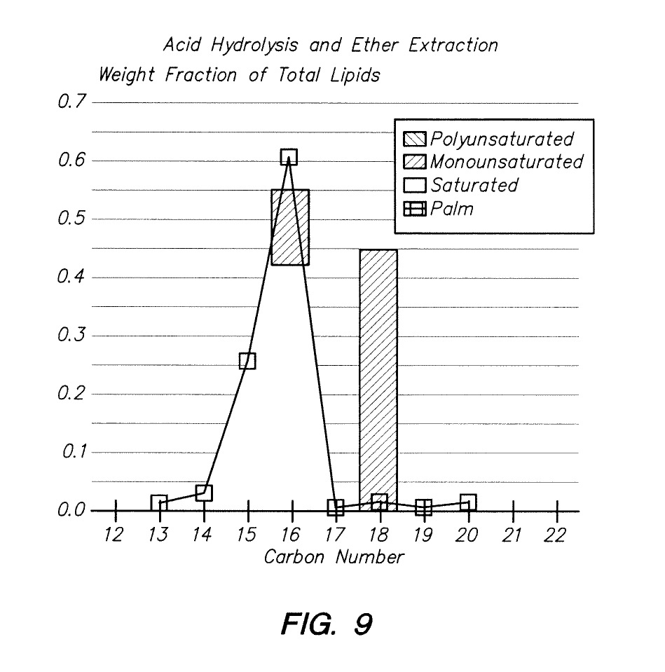

### 5. DETAILED DESCRIPTION OF THE INVENTION

The present invention relates to two important aspects of using algae to produce biofuel—cost effectiveness and energy efficiency. The supply and cost of nutrients for growing algae, and the expenditure of energy to harvest algae are often underestimated. Existing technologies for producing biofuel from algae are too expensive and inefficient when operated at a scale that is required to displace petrodiesel in the market.

In one aspect, the invention provides an integrated approach to grow algae and fish in the same system. The environmental conditions of the systems of the invention emulates certain aspects of an ecological system, preferably an ecological system that exist in the same general location as the system of the invention. The systems of the invention are more stable than monoculture, or algae culture that involves introduced non-native species.

Algae capture solar energy by photosynthesis to produce biomass. The biomass comprising lipids, among other valuable products, is a source of biofuel. The nutrients required by algae include carbon, nitrogen, phosphorous, and a host of micronutrients. On a mass basis, to make 100 units of algae, about 200 units of carbon dioxide (CO₂), 5-10 units of nitrogen (N), and 0.5-1 units of phosphorous (P) are needed. Commercial carbon dioxide cost $500/mt and would be prohibitively expensive for biofuels production, i.e., costing $250/mt of biomass or over $100/bbl of oil. With recent costs of $320/mt for ammonia and $318/mt for diammonium phosphate, the nitrogen and phosphorus would cost nominally $30/mt for dried algae and $15-30/bbl of algal oil, assuming 20-40% lipids in the algae. With oil trading at $60-80/bbl recently, the cost of the nutrients is cost-prohibitive if purchased as commercial fertilizer. As in the production of food crops in the U.S., fertilizer is often the most significant cost. The inventors recognize that recycling and recovery of nutrients from the environment and/or other sources can be advantageously adopted in the methods of culturing algae to reduce the cost of nutrients.

To illustrate the scale of the challenge, it has been estimated that stationary sources of carbon dioxide (such as power plants for electricity production, refineries, chemicals manufacturers, cement factories, and the like) in the U.S. produced CO₂ at the rate of 3.3×10⁹ tons per year in 2008. If all such CO₂ were used by algae to produce biofuel, assuming the above mass ratios and that 40% of algal biomass is lipid, the amount of biofuel would just meet the entire annual oil consumption of the United States at about 200 billion gallon/year. With respect to nitrogen, the rate of consumption was 14×10⁶ ton of ammonia per year in 2004. If all such N were used by algae to produce biofuel, assuming the above mass ratios (i.e., 5% N) and that 40% of algal biomass is lipid, the amount of biofuel produced would be equivalent to 30 billion gallons of oil per year which is only 15% of the U.S. consumption. For phosphorous, 30×10⁶ tons of P₂O₅ were consumed in 2007. Assuming the same set of mass ratios (i.e., 0.5% P) and lipid content, this amount of P would be present in about 300 billion gallons of oil, which is 150% of the U.S. consumption. Essentially, the inventors believe that any meaningful production of algae (>5% of U.S. oil needs) using commercial fertilizer will directly compete with the agricultural industry for the limited supply of fertilizer.

In one embodiment of the invention, methods for growing algae, including microalgae, in a body of water shared with a fish culture operation are provided. The algae are grown under conditions that tend to increase the number of algal cells, and/or cellular biomass. Such conditions result from the presence of the plurality of fish and can be controlled by the systems of the invention. The methods further comprise applying stress to the algae to induce lipid biosynthesis and accumulation. The presence of fish modifies the environmental conditions in the water to favor algal growth. The algal growth conditions that can be modified by fish, include but are not limited to, nitrogen content (e.g., as determined by urea concentration), phosphorous content, transparency, turbidity, quality of light exposure, intensity of light exposure, free or dissolved carbon dioxide, biological oxygen demand (BOD), chemical oxygen demand (COD), dissolved oxygen, photoperiod, and zooplankton density. Without being bound by any theory, the fishes that are co-cultured in the operation are useful for fertilizing an algal culture with metabolic wastes, providing agitation of the algal culture, and maintaining stability of the algal culture. The term “stability” refers to the state of an algal culture over a period of time, wherein the total number of algal cells, the number of different algal species, the number of particular species of algal cells (including the absence of algal species not previously present in the culture), overall growth rate, the growth rates of particular algal species, overall lipid yield, lipid yield from particular algal species, or the number of other aquatic organisms (including but not limited to fishes), is predictable or controllable, or remains relatively constant.

To boost the yield of biofuel, the algae are exposed to stress that induces the production and accumulation of lipids. Stress is any change in environmental condition that results in a metabolic imbalance and requires metabolic adjustments before a new steady state of growth can be established. Many types of stress, referred to herein individually as a “stressor” can be applied to the algae culture. Non-limiting examples of stress include changes in water quality, light quality, illumination period, and population density. The lipids and/or biomass yield of the growing algae can be monitored to assess whether a stressor is effective in inducing lipid accumulation. To boost yield, the algae may be cultured under stress for a prescribed period of time, or the algae culture may be subjected to a different stressor or, cultured under stress just before harvesting. The algae may be separated from the fishes, and/or concentrated prior to being exposed to a stressor. The algae are then harvested and used to produce algal oil by techniques known in the art, including but not limited to dewatering, pulverizing and solvent extraction.

In certain embodiments of the invention, the selected fish species used in the invention may ingest algae but do not use the algae as a primary source of food, such as when herbivorous or omnivorous fishes are used. The fishes cultured in the system can be sold, as animal feed or human food depending on the fish species and the market. However, the invention is distinguishable from aquaculture operations, such as a fish farm, wherein fish is the product of such operations. The systems and methods of the invention are designed for and preferably optimized for the production of algae which are different from those set up for culturing fish.

Algae inhabit all types of aquatic environment, including but not limited to freshwater, marine, and brackish environment, in all climatic regions, such as tropical, subtropical, temperate, and polar. Accordingly, the invention can be practiced with algae and fishes in any of such aquatic environments and climatic regions. The invention can be practiced in many parts of the world, such as but not limited to the coasts, the contiguous zones, the territorial zones, and the exclusive economic zones of the United States. For example, a system of the invention can be established in a body of water located near the coasts of Gulf of Mexico, or in the Gulf of Mexico basin, Northeast Gulf of Mexico, South Florida Continental Shelf and Slope, Campeche Bank, Bay of Campeche, Western Gulf of Mexico, and Northwest Gulf of Mexico.

The algae and the fishes that are used in the methods of the invention are described in Section 5.1 and 5.2 respectively. As used herein the term “system” refers to the installations for practicing the methods of the invention. The term “aquatic composition” is used interchangeably with the term “culture media” to refer to the water used in the systems of the invention, which, unless otherwise stated, comprises nutrients and dissolved gases required for the growth of algae. The methods and systems of the invention for culturing algae are described in Section 5.3.

Technical and scientific terms used herein have the meanings commonly understood by one of ordinary skill in the art to which the present invention pertains, unless otherwise defined. Reference is made herein to various equipment, technologies and methodologies known to those of skill in the art. Publications and other materials setting forth such known equipment, technologies and methodologies to which reference is made are incorporated herein by reference in their entireties as though set forth in full. The practice of the invention will employ, unless otherwise indicated, equipment, methodologies and techniques of chemical engineering, biology, ecology, and the fishery and aquaculture industries, which are within the skill of the art. Such equipment, technologies and methodologies are explained fully in the literature, e.g., Aquaculture Engineering, Odd-Ivar Lekang, 2007, Blackwell Publishing Ltd.; Handbook of Microalgal Culture, edited by Amos Richmond, 2004, Blackwell Science; Limnology: Lake and River Ecosystems, Robert G. Wetzel, 2001, Academic Press, each of which are incorporated by reference in their entireties.

As used herein, “a” or “an” means at least one, unless clearly indicated otherwise. The term “about,” as used herein, unless otherwise indicated, refers to a value that is no more than 20% above or below the value being modified by the term. For clarity of disclosure, and not by way of limitation, the detailed description of the invention is divided into the subsections which follow.

### 5.1. Algae

As used herein the term “algae” refers to any organisms with chlorophyll and a thallus not differentiated into roots, stems and leaves, and encompasses prokaryotic and eukaryotic organisms that are photoautotrophic or photoauxotrophic. The term “algae” includes macroalgae (commonly known as seaweed) and microalgae. For certain embodiments of the invention, algae that are not macroalgae are preferred. The terms “microalgae” and “phytoplankton”, used interchangeably herein, refer to any microscopic algae, photoautotrophic or photoauxotrophic eukaryotes (such as, protozoa), photoautotrophic or photoauxotrophic prokaryotes, and cyanobacteria (commonly referred to as blue-green algae and formerly classified as Cyanophyceae). The use of the term “algal” also relates to microalgae and thus encompasses the meaning of “microalgal.” The term “algal composition” refers to any composition that comprises algae, and is not limited to the body of water or the culture in which the algae are cultivated. An algal composition can be an algal culture, a concentrated algal culture, or a dewatered mass of algae, and can be in a liquid, semi-solid, or solid form. A non-liquid algal composition can be described in terms of moisture level or percentage weight of the solids. An “algal culture” is an algal composition that comprises live algae.

The microalgae of the invention are also encompassed by the term “plankton” which includes phytoplankton, zooplankton and bacterioplankton. For certain embodiments of the invention, an algal composition or a body of water comprising algae that is substantially depleted of zooplankton is preferred since many zooplankton consume phytoplankton. However, it is contemplated that many aspects of the invention can be practiced with a planktonic composition, without isolation of the phytoplankton, or removal of the zooplankton or other non-algal planktonic organisms. The methods of the invention can be used with a composition comprising plankton, or a body of water comprising plankton.

The algae of the invention can be a naturally occurring species, a genetically selected strain, a genetically manipulated strain, a transgenic strain, or a synthetic algae. Preferably, the algae bears at least a beneficial trait, such as but not limited to, increased growth rate, lipid accumulation, favorable lipid composition, adaptation to the culture environment, and robustness in changing environmental conditions. It is desirable that the algae accumulate excess lipids and/or hydrocarbons. The algae in an algal composition of the invention may not all be cultivable under laboratory conditions. It is not required that all the algae in an algal composition of the invention be taxonomically classified or characterized in order to for the composition be used in the present invention. Algal compositions, including algal cultures, can be distinguished by the relative proportions of taxonomic groups that are present.

The algae that are cultured or harvested by the methods of the invention either use light (autotrophic) or organic compounds (heterotrophic) as its energy source. The algae can be grown under the sunlight or artificial light. In addition to using mass per unit volume (such as mg/l or g/l), chlorophyll a is a commonly used indicator of algal biomass. However, it is subjected to variability of cellular chlorophyll content (0.1 to 9.7% of fresh algal weight) depending on algal species. An estimated biomass value can be calibrated based on the chlorophyll content of the dominant species within a population. Published correlation of chlorophyll a concentration and biomass value can be used in the invention. Generally, chlorophyll a concentration is to be measured within the euphotic zone of a body of water. The euphotic zone is a photosynthetically active layer where the light intensity exceeds 1% of that at the surface.

Depending on the latitude of a site of the system of the invention, algae obtained from tropical, subtropical, temperate, polar or other climatic regions are used in the invention. Endemic or indigenous algal species are generally preferred over introduced species where an open culturing system is used. Endemic or indigenous algae may be enriched or isolated from local water samples obtained at or near the site of the system. It is advantageous to use algae and fishes from a local aquatic trophic system in the methods of the invention. Algae, including microalgae, inhabit many types of aquatic environment, including but not limited to freshwater (less than about 0.5 parts per thousand (ppt) salts), brackish (about 0.5 to about 31 ppt salts), marine (about 31 to about 38 ppt salts), and briny (greater than about 38 ppt salts) environment. Any of such aquatic environments, freshwater species, marine species, and/or species that thrive in varying and/or intermediate salinities or nutrient levels, can be used in the invention. The algae in an algal composition of the invention can be obtained initially from environmental samples of natural or man-made environments, and may contain a mixture of prokaryotic and eukaryotic organisms, wherein some of the species may be unidentified. Freshwater filtrates from rivers, lakes; seawater filtrates from coastal areas, oceans; water in hot springs or thermal vents; and lake, marine, or estuarine sediments, can be used to source the algae. The samples may also be collected from local or remote bodies of water.

One or more species of algae are present in the algal composition of the invention. In one embodiment of the invention, the algal composition is a monoculture, wherein only one species of algae is grown. However, in many open culturing systems, it may be difficult to avoid the presence of other algae species in the water. Accordingly, a monoculture may comprise about 0.1% to 2% cells of algae species other than the intended species, i.e., up to 98% to 99.9% of the algal cells in a monoculture are of one species. In certain embodiments, the algal composition comprise an isolated species of algae, such as an axenic culture. In another embodiment, the algal composition is a mixed culture that comprises more than one species of algae, i.e., the algal culture is not a monoculture. Such a culture can be prepared by mixing different algal cultures or axenic cultures. In certain embodiments, the algal composition can also comprise zooplankton, bacterioplankton, and/or other planktonic organisms. In certain embodiments, an algal composition comprising a combination of different batches of algal cultures is used in the invention. The algal composition can be prepared by mixing a plurality of different algal cultures. The different taxonomic groups of algae can be present in defined proportions. A microalgal composition of the invention can comprise predominantly microalgae of a selected size range, such as but not limited to, below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm.

A mixed algal composition of the invention comprises one or several dominant species of macroalgae and/or microalgae. Microalgal species can be identified by microscopy and enumerated by counting visually or optically, or by techniques such as but not limited to microfluidics and flow cytometry, which are well known in the art. A dominant species is one that ranks high in the number of algal cells, e.g., the top one to five species with the highest number of cells relative to other species. Microalgae occur in unicellular, filamentous, or colonial forms. The number of algal cells can be estimated by counting the number of colonies or filaments. Alternatively, the dominant species can be determined by ranking the number of cells, colonies and/or filaments. This scheme of counting may be preferred in mixed cultures where different forms are present and the number of cells in a colony or filament is difficult to discern. In a mixed algal composition, the one or several dominant algae species may constitute greater than about 10%, about 20%, about 30%, about 40%, about 50%, about 60%, about 70%, about 80%, about 90%, about 95%, about 97%, about 98% of the algae present in the culture. In certain mixed algal composition, several dominant algae species may each independently constitute greater than about 10%, about 20%, about 30%, about 40%, about 50%, about 60%, about 70%, about 80% or about 90% of the algae present in the culture. Many other minor species of algae may also be present in such composition but they may constitute in aggregate less than about 50%, about 40%, about 30%, about 20%, about 10%, or about 5% of the algae present. In various embodiments, one, two, three, four, or five dominant species of algae are present in an algal composition. Accordingly, a mixed algal culture or an algal composition can be described and distinguished from other cultures or compositions by the dominant species of algae present. An algal composition can be further described by the percentages of cells that are of dominant species relative to minor species, or the percentages of each of the dominant species. An algal composition can also be described by the dominant species identifiable within a certain size class, e.g., below 2000 μm, about 200 to 2000 μm, above 200 μm, below 200 μm, about 20 to 2000 μm, about 20 to 200 μm, above 20 μm, below 20 μm, about 2 to 20 μm, about 2 to 200 μm, about 2 to 2000 μm, below 2 μm, about 0.2 to 20 μm, about 0.2 to 2 μm or below 0.2 μm. It is to be understood that mixed algal cultures or compositions having the same genus or species of algae may be different by virtue of the relative abundance of the various genus and/or species that are present.

Microalgae are preferably used in many embodiments of the invention; while macroalgae are less preferred in certain embodiments. In specific embodiments, algae of a particular taxonomic group, e.g., a particular genera or species, may be less preferred in a culture. Such algae, including one or more that are listed below, may be specifically excluded as a dominant species in a culture or composition. However, it should also be understood that in certain embodiments, such algae may be present as a contaminant, a non-dominant group or a minor species, especially in an open system. Such algae may be present in negligent numbers, or substantially diluted given the volume of the culture or composition. The presence of such algal genus or species in a culture, a composition or a body of water is distinguishable from cultures, composition or bodies of water where such algal genus or species are dominant, or constitute the bulk of the algae. The composition of an algal culture or a body of water in an open culturing system is expected to change according to the climate or the four seasons, for example, the dominant species in one season may not be dominant in another season. An algal culture at a particular geographic location or a range of latitudes can therefore be more specifically described by season, i.e., spring composition, summer composition, fall composition, and winter composition; or by any one or more calendar months, such as but not limited to, from about December to about February, or from about May to about September.

In various embodiments, one or more species of algae belonging to the following phyla can be cultured according to the methods of the invention: Cyanobacteria, Cyanophyta, Prochlorophyta, Rhodophyta, Glaucophyta, Chlorophyta, Dinophyta, Cryptophyta, Chrysophyta, Prymnesiophyta (Haptophyta), Bacillariophyta, Xanthophyta, Eustigmatophyta, Rhaphidophyta, and Phaeophyta. In certain embodiments, algae in multicellular or filamentous forms, such as seaweeds or macroalgae, many of which belong to the phyla Phaeophyta or Rhodophyta, are less preferred. In many embodiments, algae that are microscopic, are preferred. Many such microalgae occurs in unicellular or colonial form.

In certain embodiments, the algal culture or the algal composition of the invention comprises cyanobacteria (also known as blue-green algae) from one or more of the following taxonomic groups: Chroococcales, Nostocales, Oscillatoriales, Pseudanabaenales, Synechococcales, and Synechococcophycideae. Non-limiting examples include Gleocapsa, Pseudoanabaena, Oscillatoria, Microcystis, Synechococcus and Arthrospira species.

In certain embodiments, the algal culture or the algal composition of the invention comprises algae from one or more of the following taxonomic classes: Euglenophyceae, Dinophyceae, and Ebriophyceae. Non-limiting examples include Euglena species and the freshwater or marine dinoflagellates.

In certain embodiments, the algal culture or the algal composition of the invention comprises green algae from one or more of the following taxonomic classes: Micromonadophyceae, Charophyceae, Ulvophyceae and Chlorophyceae. Non-limiting examples include species of Borodinella, Chlorella (e.g., C. ellipsoidea), Chlamydomonas, Dunaliella (e.g., D. salina, D. bardawil), Franceia, Haematococcus, Oocystis (e.g., O. parva, O. pustilla), Scenedesmus, Stichococcus, Ankistrodesmus (e.g., A. falcatus), Chlorococcum, Monoraphidium, Nannochloris and Botryococcus (e.g., B. braunii). In certain embodiments, Chlamydomonas reinhardtii are less preferred.

In certain embodiments, the algal culture or the algal composition of the invention comprises golden-brown algae from one or more of the following taxonomic classes: Chrysophyceae and Synurophyceae. Non-limiting examples include Boekelovia species (e.g. B. hooglandii) and Ochromonas species.

In certain embodiments, the algal culture or the algal composition in the invention comprises freshwater, brackish, marine, or briny diatoms from one or more of the following taxonomic classes: Bacillariophyceae, Coscinodiscophyceae, and Fragilariophyceae. Preferably, the diatoms are photoautotrophic or auxotrophic. Non-limiting examples include Achnanthes (e.g., A. orientalis), Amphora (e.g., A. coffeiformis strains, A. delicatissima), Amphiprora (e.g., A. hyaline), Amphipleura, Chaetoceros (e.g., C. muelleri, C. gracilis), Caloneis, Camphylodiscus, Cyclotella (e.g., C. cryptica, C. meneghiniana), Cricosphaera, Cymbella, Diploneis, Entomoneis, Fragilaria, Hantschia, Gyrosigma, Melosira, Navicula (e.g., N. acceptata, N. biskanterae, N. pseudotenelloides, N. saprophila), Nitzschia (e.g., N. dissipata, N. communis, N. inconspicua, N. pusilla strains, N. microcephala, N. intermedia, N. hantzschiana, N. alexandrina, N. quadrangula), Phaeodactylum (e.g., P. tricornutum), Pleurosigma, Pleurochrysis (e.g., P. carterae, P. dentata), Selenastrum, Surirella and Thalassiosira (e.g., T. weissflogii).

In certain embodiments, the algal culture or the algal composition of the invention comprises planktons including microalgae that are characteristically small with a diameter in the range of 1 to 10 μm, or 2 to 4 μm. Many of such algae are members of Eustigmatophyta, such as but not limited to Nannochloropsis species (e.g. N. salina).

In certain embodiments, the algal culture or the algal composition of the invention comprises one or more algae from the following groups: Coelastrum, Chlorosarcina, Micractinium, Porphyridium, Nostoc, Closterium, Elakatothrix, Cyanosarcina, Trachelamonas, Kirchneriella, Carteria, Crytomonas, Chlamydamonas, Planktothrix, Anabaena, Hymenomonas, Isochrysis, Pavlova, Monodus, Monallanthus, Platymonas, Pyramimonas, Stephanodiscus, Chroococcus, Staurastrum, Netrium, and Tetraselmis.

In certain embodiments, any of the above-mentioned genus and species of algae may independently be less preferred as a dominant species in, or be excluded from, an algal composition of the invention.

### 5.2 Fishes

Fishes described in this section can be used in systems and methods of the invention for culturing algae described in the previous section. Conventional fish hatcheries and fish farming techniques known in the art can be applied to implement this aspect of the systems and methods of the invention, see for example, Chapters 10, 13, 15 in Aquaculture Engineering, Odd-Ivar Lekang, 2007, Blackwell Publishing Ltd.

As used herein, the term fish refers to a member or a group of the following classes: Actinopteryii (i.e., ray-finned fish) which includes the division Teleosteri (also known as the teleosts), Chondrichytes (e.g., cartilaginous fish), Myxini (e.g., hagfish), Cephalospidomorphi (e.g., lampreys), and Sarcopteryii (e.g., coelacanths). The teleosts comprise at least 38 orders, 426 families, and 4064 genera. Some teleost families are large, such as Cyprinidae, Gobiidae, Cichlidae, Characidae, Loricariidae, Balitoridae, Serranidae, Labridae, and Scorpaenidae. In many embodiments, the invention involves bony fishes, such as the teleosts, and/or cartilaginous fishes.

When referring to a plurality of organisms, the term “fish” is used interchangeably with the term “fishes” regardless of whether one or more than one species are present, unless clearly indicated otherwise. Fishes useful for the invention can be obtained from fish hatcheries or collected from the wild. The fishes may be fish fry, juveniles, fingerlings, or adult/mature fish. In certain embodiments of the invention, juveniles that have metamorphosed are used. By “fry” it is meant a recently hatched fish that has fully absorbed its yolk sac, while by “juvenile” or “fingerling” it is meant a fish that has not recently hatched but is not yet an adult. In certain embodiments of the invention, fry and/or juveniles can be used. The fishes may reproduce in an enclosure (e.g., growth enclosure or fish enclosure) within the system and not necessarily in a fish hatchery. Any fish aquaculture techniques known in the art can be used to stock, maintain, reproduce, and gather the fishes used in the invention. Depending on the local environment and the type of fish used, the fish can be introduced at various density from about 50 to 100, about 100 to 300, about 300 to 600, about 600 to 900, about 900 to 1200, and about 1200 to 1500 individuals per m².

One or more species of fish can be used in the growth enclosure for culturing algae. In one embodiment of the invention, the population of fish comprises only one species of fish. In another embodiment, the fish population is mixed and thus comprises one or several major species of fish. A major species is one that ranks high in the head count, e.g., the top one to five species with the highest head count relative to other species. The one or several major fish species may constitute greater than about 10%, about 20%, about 30%, about 40%, about 50%, about 60%, about 70%, about 75%, about 80%, about 90%, about 95%, about 97%, about 98% of the fish present in the population. In certain embodiments, several major fish species may each constitute greater than about 10%, about 20%, about 30%, about 40%, about 50%, about 60%, about 70%, or about 80% of the fish present in the population. In various embodiments, one, two, three, four, five major species of fish are present in a population of fishes. Accordingly, a mixed fish population or culture can be described and distinguished from other populations or cultures by the major species of fish present. The population or culture can be further described by the percentages of the major and minor species, or the percentages of each of the major species. It is to be understood that mixed cultures having the same genus or species may be different by virtue of the relative abundance of the various genus and/or species present.

Fish inhabits most types of aquatic environment, including but not limited to freshwater, brackish, marine, and briny environments. As the present invention can be practiced in any of such aquatic environments, any freshwater species, stenohaline species, euryhaline species, marine species, species that grow in brine, and/or species that thrive in varying and/or intermediate salinities, can be used. Fishes from tropical, subtropical, temperate, polar, and/or other climatic regions can be used. Fishes that live within the following temperature ranges can be used: below 10° C., 9° C. to 18° C., 15° C. to 25° C., 20° C. to 32° C. In one embodiment, fishes indigenous to the region at which the methods of the invention are practiced, are used. Preferably, fishes from the same climatic region, same salinity environment, or same ecosystem, as the algae are used.

In an aquatic environment, fish occupies various trophic levels, such as piscivores (carnivores), herbivores, planktivores, detritivores, and omnivores. Many species of planktivores develop specialized anatomical structures to enable filter feeding, e.g., gill rakers and gill lamellae. Generally, the size of such structures relative to the dimensions of the plankton in the water, including microalgae, affects the diet of a planktivore. Fish having more closely spaced gill rakers with specialized secondary structures to form a sieve are typically phytoplanktivores. Others having widely spaced gill rakers with secondary barbs are generally zooplanktivores. In the case of piscivores, the gill rakers are generally reduced to barbs. Gut content analysis can determine the diet of an organism used in the invention. Techniques for analysis of gut content of fish are known in the art. As used herein, a planktivore is a phytoplanktivore if a population of the planktivore, reared in water with non-limiting quantities of phytoplankton and zooplankton, has on average more phytoplankton than zooplankton in the gut, for example, greater than 50%, 60%, 70%, 80%, or 90%. Under similar conditions, a planktivore is a zooplantivore if the population of the planktivore has on average more zooplankton than phytoplankton in the gut, for example, greater than 50%, 60%, 70%, 80%, or 90%. Certain fish can consume a broad range of food or can adapt to a diet offered by the environment. Accordingly, it is preferable that the fish are cultured in a system of the invention before undergoing a gut content analysis.

The selection of fishes for use in the culturing methods of the invention depends on a number of factors, the foremost of which is the compatibility of the cultured algae and the fishes. Preferably, the algae culture grows well using the metabolic wastes (dissolved and/or solid waste) produced by the selected fishes, thereby reducing the need to fertilize the water or to change the water. Preferably, the population of fishes is self-sustaining in the system of the invention and does not require extensive fish husbandry efforts to promote reproduction and to rear the juveniles. The methods of the invention can employ species of fishes that are used as human food or animal feed, to offset the cost of operating the algae culture. Fishes that do not use phytoplankton as a major source of energy are preferred in the culturing systems and methods of the invention. Fishes that commingle with algae in the growth enclosure in the culturing methods are preferably not phytoplanktivores. Herbivores that consume macroalgae or aquatic vascular plants can be used where microalgae are being cultured. Detritivores or piscivores are preferably used in the methods of culturing algae of the invention. In some embodiments of the invention, the population of fish in the growth enclosure comprises predominantly detritivores. In some embodiments of the invention, the population of fish comprises predominantly omnivores. In some embodiments of the invention, the population of fish comprises predominantly omnivores. In some embodiments of the invention, the population of fish comprises predominantly zooplanktivores. In some embodiments of the invention, the population of fish comprises predominantly piscivores. The predominance of one type of fish as defined by their trophic behavior over another type in a population of fishes can be defined by percentage head count as described above for describing major fish species in a population (e.g., about 90% piscivores and 10% omnivores; or about 80% detritivores, 20% herbivores).

Fishes from different taxonomic groups can be used in the growth enclosure or fish enclosure. It should be understood that, in various embodiments, fishes within a taxonomic group, such as a family or a genus, can be used interchangeably in various methods of the invention. The invention is described below using common names of fish groups and fishes, as well as the scientific names of exemplary species. Databases, such as FishBase by Froese, R. and D. Pauly (Ed.), World Wide Web electronic publication, www.fishbase.org, version (06/2008), provide additional useful fish species within each of the taxonomic groups that are useful in the invention. It is contemplated that one of ordinary skill in art could, consistent with the scope of the present invention, use the databases to specify other species within each of the described taxonomic groups for use in the methods of the invention. The selected fishes should grow well in water of a salinity which is similar to that of the algal culture, so as to reduce the need to change water when the algae is brought to the fishes. For an open pond system, it may be preferable to use endemic species of fishes.

In certain embodiments of the invention, the fish population comprises fishes in the order Acipeneriformes that do not feed on phytoplanktons or use phytoplanktons as a major source of energy, such as but not limited to, sturgeons (trophic level 3) e.g., Acipenser species, and Huso huso.

In certain embodiments of the invention, the fish population comprises fishes in the order Clupiformes that do not feed on phytoplanktons or use phytoplanktons as a major source of energy. The order Clupiformes includes the following families: Chirocentridae, Clupeidae (menhadens, shads, herrings, sardines, hilsa), Denticipitidae, Engraulidae (anchovies). Exemplary members within the order Clupiformes include but not limited to, the menhadens (Brevoortia species), e.g, Ethmidium maculatum, Brevoortia aurea, Brevoortia gunteri, Brevoortia smithi, Brevoortia pectinata, Gulf menhaden (Brevoortia patronus), and Atlantic menhaden (Brevoortia tyrannus); the shads, e.g., Alosa alosa, Alosa alabamae, Alosa fallax, Alosa mediocris, Alosa sapidissima, Alosa mediocris, Dorosoma petenense; the herrings, e.g., Etrumeus teres, Harengula thrissina, Pacific herring (Clupea pallasii pallasii), Alosa aestivalis, Ilisha africana, Ilisha elongata, Ilisha megaloptera, Ilisha melastoma, Ilisha pristigastroides, Pellona ditchela, Opisthopterus tardoore, Nematalosa come, Alosa aestivalis, Alosa chrysochloris, freshwater herring (Alosa pseudoharengus), Arripis georgianus, Alosa chrysochloris, Opisthonema libertate, Opisthonema oglinum, Atlantic herring (Clupea harengus), Baltic herring (Clupea harengus membras); the sardines, e.g., Ilisha species, Sardinella species, Amblygaster species, Opisthopterus equatorialis, Sardinella aurita, Pacific sardine (Sardinops sagax), Harengula clupeola, Harengula humeralis, Harengula thrissina, Harengula jaguana, Sardinella albella, Sardinella Janeiro, Sardinella fimbriata, oil sardine (Sardinella longiceps), and European pilchard (Sardina pilchardus); the hilsas, e.g., Tenuolosa species and the anchovies, e.g., Anchoa species, Engraulis species, Thryssa species, anchoveta (Engraulis ringens), European anchovy (Engraulis encrasicolus), Australian anchovy (Engraulis australis), Setipinna phasa, Coilia dussumieri.

In certain embodiments of the invention, the fish population comprises fishes in the superorder Ostariophysi, which include the order Gonorynchiformes, order Siluriformes, and order Cypriniformes, that do not feed on phytoplanktons or use phytoplanktons as a major source of energy. Non-limiting examples of fishes in this superorder include catfishes, barbs, carps, danios, goldfishes, loaches, shiners, minnows, and rasboras. The catfishes, such as channel catfish (Ictalurus punctatus), blue catfish (Ictalurus furcatus), catfish hybrid (Clarias macrocephalus), Ictalurus pricei, Pylodictis olivaris, Brachyplatystoma vaillantii, Pinirampus pirinampu, Pseudoplatystoma tigrinum, Zungaro zungaro, Platynematichthys notatus, Ameiurus catus, Ameiurus melas are detritivores. Carps are freshwater herbivores and detritus feeders, e.g., common carp (Cyprinus carpio), Chinese carp (Cirrhinus chinensis), black carp (Mylopharyngodon piceus), silver carp (Hypophthalmichthys molitrix), bighead carp (Aristichthys nobilis) and grass carp (Ctenopharyngodon idella). Shiners includes members of Luxilus, Cyprinella and Notropis genus, such as but not limited to, Luxilus cornutus, Notropis jemezanus, Cyprinella callistia. Other useful herbivores and detritus feeders are members of the Labeo genus, such as but not limited to, Labeo angra, Labeo ariza, Labeo bata, Labeo boga, Labeo boggut, Labeo porcellus, Labeo kawrus, Labeo potail, Labeo calbasu, Labeo gonius, Labeo pangusia, and Labeo caeruleus.

In certain embodiments of the invention, the fish population comprises fishes in the superorder Protacanthopterygii that do not feed on phytoplanktons or use phytoplanktons as a major source of energy. This superorder includes the order Salmoniformes and order Osmeriformes. Non-limiting examples of fishes in this superorder include the salmons, e.g., Oncorhynchus species, Salmo species, Arripis species, Brycon species, Eleutheronema tetradactylum, Atlantic salmon (Salmo salar), red salmon (Oncorhynchus nerka), and Coho salmon (Oncorhynchus kisutch); and the trouts, e.g., Oncorhynchus species, Salvelinus species, Cynoscion species, cutthroat trout (Oncorhynchus clarkii), and rainbow trout (Oncorhynchus mykiss); which are trophic level 3 carnivorous fish.

In certain embodiments of the invention, the fish population comprises fishes in the superorder Acanthopterygii, that do not feed on phytoplanktons or use phytoplanktons as a major source of energy. The superorder includes the order Mugiliformes, Pleuronectiformes, and Perciformes. Non-limiting examples of this superorder are flatfishes which are carnivorous; the anabantids; the centrarchids (e.g., bass and sunfish); the cichlids, the gobies, the gouramis, mackerels, perches, scats, whiting, snappers, groupers, barramundi, drums wrasses, and tilapias (Oreochromis sp.). Examples of tilapias include but is not limited to nile tilapia (Oreochromis niloticus), red tilapia (O. mossambicus×O. urolepis hornorum), mango tilapia (Sarotherodon galilaeus).

Many species of fishes are farmed or captured for human consumption, making animal feed, including aquaculture feed, and a variety of other oleochemical-derived products, such as paints, linoleum, lubricants, soap, insecticides, and cosmetics. The methods of the invention can employ species of fishes that are otherwise used as human food, animal feed, or oleochemical feedstocks. Depending on the economics, some of the fishes produced by the present method can be sold as human food, animal feed or oleochemical feedstock. In certain embodiments, the fishes used in the present invention are not suitable for making animal feed, human food, or oleochemical feedstock.

Transgenic fish and genetically improved fish can also be used in the culturing systems and methods of the invention. The term “genetically improved fish” refers herein to a fish that is genetically predisposed to having a higher growth rate than a wild type fish, when they are cultured under the same conditions. Such fishes can be obtained by traditional breeding techniques or by transgenic technology. Over-expression or ectopic expression of a piscine growth hormone transgene in a variety of fishes resulted in enhanced growth rate. For example, the growth hormone genes of Chinook salmon, Sockeye salmon, tilapia, Atlantic salmon, grass carp, and mud loach have been used in creating transgenic fishes (Zbikows ka, Transgenic Research, 12:379-389, 2003; Guan et al., Aquaculture, 284:217-223, 2008).

### 5.3 Methods and Systems

In one aspect of the invention, systems and methods for growing algae to produce biofuel are provided. The culturing systems of the invention comprise one or more water-containing enclosures for growing algae and fishes, means for culturing the algae, and means for growing the fishes. The culturing systems can further comprise means for controlling the conditions of the aquatic environment in the system, means for concentrating the algae mechanically and/or means for harvesting the algae mechanically. The culturing systems can further comprise means for converting algal biomass into energy feedstocks. According to the invention, the algae as described in Section 5.1 and the fishes as described in Section 5.2 are cultured for a period of time in the same volume of water where the algae reproduce and grow.

The algae and fishes are considered to be cultured in an aquatic composition or in the same body of water where at least one quality of the water that is modified by the presence of the fishes enable the algae to grow more efficiently than in the absence of the fishes. In certain embodiments of the invention, the algae culture requires less or no fertilizer to sustain growth at a particular growth rate (e.g., 5%, 10%, 15%, 20%, 25%, 30%, 40%, 50%, 60%, 70%, 80%, 90%, 95% less nitrogen and/or phosphorous, or organic and/or inorganic fertilizer than a control system or a natural system in the same environment). In certain embodiments of the invention, the algae culture requires a lower input of energy required to provide adequate mixing (e.g., 5%, 10%, 15%, 20%, 25%, 30%, 40%, 50% less energy used than a control system). However, it is not required that the volume of the aquatic composition in a system of the invention remains unchanged throughout the process as water comprising nutrients and/or gases may be added, water comprising waste may be removed from the system, water level may rise due to rain, change in ground water level or tide, and water may evaporate under ambient conditions. Nor is it required that the fishes and the algae be cultured in the system or in the same aquatic composition or body of water throughout the entire process.

In one embodiment of the invention, the fishes and the algae reside or commingle in the same enclosure. In another embodiment, the fishes and the algae reside in the same enclosure but the fishes are confined or caged in a zone within the enclosure. In yet another embodiment, the fishes and the algae reside in the same enclosure but the algae are confined in a space inaccessible to the fishes within the enclosure. In yet another embodiment, the fishes and a majority of the algae are physically separated in different enclosures but share the aquatic composition or the same body of water that is circulated periodically or continuously between the enclosures. In yet another embodiment, the fishes and the bulk of the algae reside in different enclosures but the algae is allowed to flow into the enclosures in which the fishes reside, and return to the initial enclosure. In yet another embodiment, the fishes and the bulk of the algae reside in different enclosures but the aquatic composition in the enclosures in which the fishes reside flows periodically or continuously to the enclosure comprising the algae.

The culture systems of the invention comprise means for culturing algae and means for culturing fishes. The means for culturing algae and means for culturing fish can be, independently, but is not limited to a water-containing enclosure on land, on coastal land (e.g., marshland, bayou), in a natural body of water (e.g., lakes), or at sea. This enclosure, referred to herein generally as a growth enclosure can be but is not limited to a raceway, rectangular tank, circular tank, partitioned tank, plastic bag, earthen pond, lined pond, channel, and artificial stream. The growth enclosure can comprise submerged or floating cages, net-pens, and such like to confine the movement of the fish inside a growth enclosure. The culturing systems further comprise means for controlling the aquatic environment in the system which include but are not limited to means for connecting the growth enclosures to each other and to other parts of the system to facilitate fluid flow, periodically, continuously, and temporarily or permanently. The connecting means can include but is not limited to channels, hoses, conduits, viaducts, and pipes. The culturing systems further comprise means for regulating the rate, direction, or both the rate and direction, of fluid flow between the growth enclosure(s) and other parts of the system. The flow regulating means can include but is not limited to pumps, valves, and gates. The flow of an aquatic composition within a system of the invention can thus be controlled. The culturing systems further comprise means for introducing fish to an enclosure, means for removing fish from an enclosure, and/or means for transferring fishes between enclosures of the systems. The enclosures of the invention can be set up according to knowledge known in the art, see, for example, Chapters 13 and 14 in Aquaculture Engineering, Odd-Ivar Lekang, 2007, Blackwell Publishing Ltd., respectively, for description of closed culturing systems and open culturing systems. Other instruments and technology for monitoring and controlling aquatic environments known in the art can be applied in the methods and systems of the invention, see, for example, in Chapter 19 of Aquaculture Engineering, Odd-Ivar Lekang, 2007, Blackwell Publishing Ltd.

The enclosures of the systems of the invention can be closed or open, or a combination of open and closed enclosures. The enclosures can be completely exposed, covered, reversibly covered, or partly covered. The communication between a closed enclosure and its immediate aquatic and/or atmospheric environment is highly controlled relative to an open enclosure. Systems comprising open enclosures can be installed with or without means for environmental controls. The size of an open enclosure of the invention can range, for example, from about 0.05 hectare (ha) to 20 ha, from about 0.25 to 10 ha, and preferably from about 1 to 5 ha. Systems comprising open enclosures that are situated on land can comprise one or more growth enclosure(s) and/or fish enclosure(s), which can be independently, ponds and/or raceways. The depth of such systems can range, for example, from about 0.3 m to 4 m, from about 0.8 m to 3 m, and from about 1 to 2 m. Raceways can be operated at shallow depths of 15 cm to 1 m. Typical dimensions for raceways are about 30:3:1 (length:width:depth) with slanted or vertical sidewalls. The systems can comprise a mix of different physical types of enclosures. The enclosures of the invention can be set up according to knowledge known in the art, see, for example, Chapters 13 and 14 in Aquaculture Engineering, Odd-Ivar Lekang, 2007, Blackwell Publishing Ltd., respectively, for description of closed culturing systems and open culturing systems.

The mode of algal culture can be a batch culture, a continuous culture, or a semi-continuous culture. A batch culture comprises providing one or more inoculations of algal cells in a volume of water in the growth enclosure at the beginning of a growing period, and when it reaches a desirable density or at the end of the growing period, harvesting the algal population. Typically, the growth of algae is characterized by a lag phase, a growth phase, and a stationary phase. The lag phase is attributed to physiological adaptation of the algal metabolism to growth. Cultures inoculated with exponentially growing algae have short lag phases and are thus desirable. Cell density increases as a function of time exponentially in the growth phase. The growth rate decreases as nutrient levels, carbon dioxide, unfavorable pH, or other environmental factors become limiting in a stationary phase culture. When a growing algae culture has outgrown the maximum carrying capacity of an enclosure, the culture can be transferred to one or several growth enclosures with a lower loading density. The initial algal culture is thereby diluted allowing the algae to grow without being limited by the capacity of an enclosure. In a continuous culture, water with nutrients and gases is continuously allowed into the growth enclosure to replenish the culture, and excess water is continuously removed while the algae in the water are harvested. The culture in the growth enclosure is maintained at a particular range of algal density or growth rate. In a semi-continuous culture, growing algae in an enclosure is harvested periodically followed by replenishment to about the original volume of water and concentrations of nutrient and gases. Continuous systems are preferred for its efficiency and economy since they are operational most of the time and require less labor to restart the culture.

Most natural land-based water sources, such as but not limited to rivers, lakes, springs and aquifers, and municipal water supply can be used as a source of water for used in the systems of the invention. Seawater from the ocean or coastal waters, artificial seawater, brackish water from coastal or estuarine regions can also be a source of water. Irrigation water, eutrophic river water, eutrophic estuarine water, eutrophic coastal water, agricultural wastewater, industrial wastewater, or municipal wastewater can also be used in the systems of the invention. Optionally, one or more effluents of the system are recycled within the system. The systems of the invention optionally comprise means for connecting the enclosures to each other, to other parts of the system and to water sources and points of disposal. The means for connecting, either temporary or permanent, facilitates fluid flow and allows fluid exchange, and can include but is not limited to a network of channels, hoses, conduits, viaducts, and pipes. The systems further comprise means for regulating the rate, direction, or both the rate and direction, of fluid flow throughout the network by standard chemical engineering techniques, such as flow of water between the enclosures and between the enclosures and other parts of the system. The flow regulating means can include but is not limited to pumps, valves, manifolds, and gates. Optionally, effluents from one or more enclosures are recycled generally within the system, or selectively to certain parts of the system.

The systems of the invention also provide means to monitor and/or control the aquatic environment of the enclosures, which includes but is not limited to means to monitor and/or control, independently or otherwise, the pH, salinity, dissolved oxygen, temperature, turbidity, nitrogen concentration, phosphorous concentration, and other conditions of the water. The enclosures of the invention can operate within the following non-limiting, exemplary water quality limits: dissolved oxygen at greater than 5 mg/L, pH 6-10 and preferably pH from 6.5-8.2 for cold water fishes and pH7.5 to 9.0 for warm water fishes; alkalinity at 10-400 mg/L CaCO₃; salinity at 0.1-3.0 g/L for stenohaline fishes and 28-35 g/L for marine fishes; less than 0.5 mg ammonia/L; less than 0.2 mg nitrite/L; and less than 10 mg/L CO₂ Equipment commonly employed in the aquaculture industry, such as thermometers, thermostats, pH meters, conductivity meters, dissolved oxygen meters, and automated controllers can be used for monitoring and controlling the aquatic environments of the system. For example, the pH of the water is preferably kept within the ranges of from about pH6 to pH9, and more preferably from about 8.2 to about 8.7. The salinity of water ranges preferably from about 12 to about 40 g/L and more preferably from 20 to 24 g/L. The temperature for seawater-based culture ranges preferably from about 16° C. to about 27° C. or from about 18° C. to about 24° C.

Generally, oxygen consumption by fish increases shortly after feeding, and water temperature regulates the rate of metabolism. The oxygen transport rate from water to fish is directly dependent on the partial oxygen pressure differences between fish blood (e.g., 50-110 mmHg) and the dissolved oxygen concentration in water (e.g., 154-158 mmHg at sea level), equilibrated to temperature and atmospheric pressure. During the day, the algae will provide oxygen whereas the fish and bacteria (via decomposition of organic matters) will provide the carbon dioxide. At night, essentially all of the organisms will respire and may require active oxygenation. The systems of the invention can comprise means for delivering a gas, or a liquid comprising a dissolved gas to the aquatic composition in the systems, which include but are not limited to hoses, pipes, pumps, valves, and manifolds. Means for delivering carbon dioxide or oxygen via aeration (e.g., bubbling or paddle wheel) or compressed gas are contemplated. Bubbles in the culture media can be formed by injecting gas, such as air, using a jet nozzle, sparger or diffuser, or by injecting water with bubbles using a venturi injector. Various techniques and means for oxygenation of water known in the art can be applied in the method of the invention, see, for example, Chapter 8 in Aquaculture Engineering, Odd-Ivar Lekang, 2007, Blackwell Publishing Ltd.

Depending on the source of water, it may be necessary to provide additional nutrients. The growth enclosures can be fertilized regularly according to conventional fishery practices. The primary macronutrients: nitrogen and phosphorus can be added as synthetic fertilizer as one of a combination of the following: anhydrous ammonia, ammonium sulfate, ammonium nitrate, urea, urea formaldehyde, urea-ammonium-nitrate (UAM) solutions, phosphoric acid, phosphorus pentoxide, diammonium phosphate (DMP), calcium super phosphate, and various N/P/K fertilizers (16-20-20, or 14-14-14), or as a natural fertilizer that can include manure from dairy farms, pig farms, poultry farms, municipal wastewater, worm castings, peat, and guano. However, less fertilizer is required in the systems of the invention than a system without fishes because they excrete metabolic waste in the enclosure.

The addition of carbon dioxide promotes photosynthesis, and helps to maintain the pH of the culture below pH 9. The source of carbon for the algae growth can either be naturally available: atmospheric CO₂, dissolved CO₂, or bicarbonate in water; or man-made: commercial CO₂ or CO₂ discharged from a stationary source, such as but not limited to, synthetic fuel plants, gasification power plants, oil recovery plants, ammonia plants, ethanol plants, oil refinery plants, anaerobic digestion units, cement plants, and fossil steam plants. Carbon dioxide, either dissolved or as bubbles, at a concentration from about 0.03% to 1%, and up to 20% volume of gas, either air or nitrogen, can be introduced into the enclosures. The CO₂ can be bubbled or sparged into the water to control the CO₂ levels either at intervals (hourly or daily), or through a feed-back control loop that continuously monitors CO₂ concentration and adds CO₂ as needed.

According to the methods of the invention, a starter culture of algae can be used to seed a growth enclosure. A starter culture can also be used to inoculate a growth enclosure periodically to maintain a stable population of the desired species. The starter culture is grown in water enclosures typically smaller than the growth enclosure, referred to herein as “inoculation enclosures.” The inoculation enclosures can be, but not limited to, one or more flasks, carboys, cylinders, plastic bags, chambers, indoor tanks, outdoor tanks, indoor ponds, and outdoor ponds, or a combination thereof. One or more inoculation enclosures can be temporarily or permanently connected to one or more growth enclosures and to each other with means for regulating fluid flow and flow direction, e.g., gate, valve. Typically, the volume of an inoculation enclosure ranges from 1 to 10 liters, 5 to 50 liters, 25 to 150 liters, 100 to 500 liters. In certain embodiments, the inoculation enclosure does not comprise fish.

For productive growth in an enclosure, the algae are exposed to light of an intensity that ranges from 1000 to 10,000 lux, preferably 2500 to 5000 lux. The photoperiod (light:dark in number of hours) ranges from about 12:12, about 14:10, about 16:8, about 18:6, about 20:4, about 22:2, and up to 24:0. The light quality (e.g, the spectrum of wavelengths), light intensity and photoperiod depend on the geographic location of the growth enclosures and the season, and may be affected by the presence of fishes, and can be controlled by artificial illumination or shading. In one aspect, mixing of water in the growth enclosure ensures that all algal cells are equally exposed to light and nutrients. Mixing is also necessary to prevent sedimentation of the algae to the bottom or to a depth where light penetration becomes limiting. Mixing also prevents thermal stratification of outdoor cultures, thus promoting temperature uniformity of the aquatic composition. Mixing is provided in part or solely by the presence of swimming fish in the growth enclosure. Where additional mixing is required, it can be provided by any mixing means, mechanical or otherwise, including but not limited to, agitation by paddle wheels and water pumps.

According to the invention, the aquatic conditions for growing algae can be controllably modified by fish in the system. In one aspect of the invention, the aquatic conditions, such as nutrient levels (e.g., N, P), are modified by increasing or decreasing the degree of mixing in the body of water, or in one or more zones within the body of water. The degree of mixing can be increased or decreased by adjusting the power supplied to the device(s), such as paddle wheel or pumps, that perform the mixing and distribution of nutrients. In a specific embodiment of the invention, fishes are confined to a zone, such as a cage, in a body of water in which the algae are cultured. Where the fishes, which can serve as a source of nutrients for algae, are localized in a zone within a body of water, controlled mixing can establish one or more nutrient gradients or a uniform nutrient level within the body of water, thereby stimulating the growth of algae or stressing the algae. Algae growing in stagnant water will consume nutrients and deplete the nutrients over a period of time, resulting in starvation and stress. Thus, methods of the invention comprise increasing or decreasing the degree of mixing in an enclosure, or in a zone within an enclosure.

It is also contemplated that the aquatic conditions, such as nutrient levels (e.g., N, P), can be controlled by confining the fish in one or more zones in an enclosure of the system, adding fish to or removing fish from an enclosure of the system, adding fish to or removing fish from one or more zone(s) within an enclosure, or changing the relative number of different species (or trophic types) of fishes within an enclosure or within a zone. Cages containing the fishes can be relocated to various zones within the body of water, or to different parts of the system. Accordingly, methods of the invention comprise increasing or decreasing the total number of fish, or the number of fish of any one or more species, in an enclosure, in a zone or a cage.

In addition to algae and fishes, in certain embodiments, the enclosures of the invention may comprise one or more additional aquatic organisms, such as but not limited to bacteria; plankton including zooplankton, such as but not limited to larval stages of fishes (i.e., ichthyoplankton), tunicates, cladocera and copepoda; crustaceans, insects, worms, nematodes, mollusks and larval forms of the foregoing organisms; and aquatic plants. This type of culture system emulates certain aspects of an ecological system. The presence of bacteria, plants, and animal species beside fishes lend additional stability to an algal culture that is maintained in the open. The fishes of the system may feed on any one of these types of organisms. These organisms can be introduced into the system or they may be present in the environment in which the culture system is established. However, planktivores graze on microalgae and are generally undesirable if present in excess in a growth enclosure of the invention. They can be removed from the water by sand filtration or by being eaten by planktivorous fishes in the enclosure. The numbers and species of planktivores, including phytoplanktivores, can be assessed by counting under a microscope using, for example, a Sedgwick-Rafter cell.

On one or more occasions during the culturing process, the cultured algae are induced by stress to accumulate lipids. In one embodiment of the invention, the algae in the growth enclosure are separated from the fish prior to exposure to stress. In another embodiment, the algae in the growth enclosure are concentrated prior to exposure to stress. In yet another embodiment, the algae can be separated from the fish and then concentrated, prior to exposure to stress. In various embodiments, the algae in the growth enclosure are exposed to one or more stressors for an interval to promote lipid production and accumulation prior to harvesting. When more than one stressors are applied, it is not required that the algae are subjected to the various stressors for the same period of time. The stressors may be applied sequentially or simultaneously. In various embodiments, the algae can be subjected to multiple rounds of concentration followed by exposure to a stressor for an interval, prior to harvesting. It should be understood that the algae may continue to grow when it is exposed to a stressor, albeit at a rate typically slower than the rate during the growth phase before the stress is applied.

Many changes in water quality can be a stressor, including but not limited to salinity, conductivity, turbidity, water temperature, nitrogen content (e.g., urea concentration), phosphorus content (e.g., orthophosphate concentration), silicon content (e.g., silicate concentration), and iron content, alkalinity. Light intensity and photoperiod can be manipulated to stress the algal culture. For certain algae, such as Nannochloropsis, the cellular content of total polyunsaturated fatty acids and total lipids is inversely related to light intensity. A shift in water temperature is a stressor that can be used to induce lipid accumulation in algae. At an optimal temperature for growth, algal cells attain minimal size, maintain low cellular carbon and nitrogen content, but multiply rapidly resulting in an increase in cell number. While at temperature above or below the optimal temperature, algal cells increase in volume and cellular content, including lipids, and algal cell division slows. Salinity is affected by a combination of the effect of rain and evaporation, and can be controlled by adding either fresh or saline water to the enclosures of the system.

Nutrient limitation is a class of stressors that can be applied to induce lipid biosynthesis and accumulation. Algae generally utilize at least 30 inorganic elements. In addition to major constituents, C, N, and P, other macronutrients include Si, S, K, Na, Fe, Mg, and Ca. The micronutrients include B, Cu, Mn, Zn, Mo, Co, V, and Se. With the exception of C, N, P, and Si, the other nutrients are generally available at sufficient levels in most water sources. Under nitrogen-limiting conditions, most algae divert the flow of fixed carbon to the biosynthesis of lipids and/or carbohydrates. Neutral lipids such as triglycerols, in particular, can become the predominant lipids in certain nitrogen-depleted algae. The amounts of lipids and carbohydrates accumulated in algae grown under nutrient limiting conditions relative to algae grown under non-limiting conditions can readily be tested by methods known in the art.

Concentration of algae in an algal culture or algal composition reduces the volume of water that has to be processed when the algae is harvested. By using a reduced volume of water and a higher concentration of algae relative to the algal culture in the growth enclosure, it would be more efficient and economical to apply a stressor to the algae. Under certain circumstances, even by concentrating and growing an algal culture to a high cell density, the algae are, by the overcrowding, induced to produce and accumulate lipids. This is caused in part because in a smaller volume of water, less nutrient and dissolved gases are available to the algal cells, while the level of metabolic waste increases. Accordingly, the density of algae in the enclosure can be monitored and adjusted, such as by maintaining the density at a constant level that is at least about two times, about three times, about five times, about 10 times, about 20 times, or about 50 times the average amount of algae normally present in a natural aquatic environment, such as a local aquatic environment in which the endemic algae species exist. An algal composition of the invention can be a concentrated algal culture or composition that comprises about 110%, 125%, 150%, 175%, 200% (or 2 times), 250%, 500% (or 5 times), 750%, 1000% (10 times) or 2000% (20 times) the amount of algae in the original culture or in a preceding algal composition. For example, the algae can be present at a concentration of greater than about 10, 25, 50, 75, 100, 250, 500, 750, 1000 mg/L, or about 10 to about 500 mg/L, about 50 to about 200 mg/L, or about 200 to 1000 mg/L. At a density that is higher than that of a natural aquatic environment, and depending on the dimensions of the enclosure and the amount of agitation, less light is available to the algae due to shading as some algae sink deeper into the enclosure.

In various embodiments of the invention, the algae can be concentrated so that the number of algal cells per unit volume increases by two, five, 10, 20, 25, 30, 40, 50, 75, 100-fold, or more. For example, the starting concentration of an algal culture can range from about 0.05 g/L, about 0.1 g/L, about 0.2 g/L, about 0.5 g/L to about 1.0 g/L. After the concentration step, the concentration of algae in an algal composition can range from at least about 0.2 g/L, about 0.5 g/L, about 1.0 g/L, about 2.0 g/L, about 5 g/L to about 10 g/L. An alternative system to assess algal concentration that measures chlorophyll-a concentration (μg/L) can be used similarly. The concentration of algae can be increased progressively by concentrating the algae in multiple stages. Starting in the growth enclosure, the algal culture is concentrated to provide an algal composition comprising algae at a density or concentration that is higher than that of the algal culture in the growth enclosure. The concentrated algal composition can be subjected to another round of concentration using the same or a different technique. Alternatively, the concentrated algal composition can be grown for an interval in an enclosure separate from the growth enclosure or in a separate zone within the growth enclosure. The zone prevents the mixing of the concentrated algae with the algae and the fish in the growth enclosure but uses the same water as in the growth enclosure. After an interval of growth at a higher density, the algae can be subjected to another round of concentration or it can be harvested. It is contemplated that the systems of the invention comprise, in the growth enclosure, one or more zones that hold the concentrated algal compositions. The concentrated algal composition can also be held in one or more separate enclosures. The methods of the invention comprise concentrating the algae in the growth enclosure for one or more rounds, wherein the output of a first or earlier rounds serve as the input of a second or successive rounds. After each round of concentration, the algae may be grown for an interval before the next round. The growth intervals are generally shorter than the period of growth in the growth enclosure. Although it is desirable to remove as much water as possible from the algae before processing, it should be understood that the concentration step does not require that the algae be dried, dewatered, or reduced to a paste or any semi-solid state. The resulting concentrated algae composition can be a solid, a semi-solid (e.g., paste), or a liquid (e.g., a suspension), and it can be stored or used to make biofuel immediately.

The concentration step can be performed serially by one or more different techniques to obtain a concentrated algal composition. Any techniques and means known in the art for concentrating the algae can be applied, including but not limited to centrifugation, filtration, sedimentation, flocculation, and foam fractionation. See, for example, Chapter 10 in Handbook of Microalgal Culture, edited by Amos Richmond, 2004, Blackwell Science, for description of downstream processing techniques. Centrifugation separates algae from the culture media and can be used to concentrate or dewater the algae. Various types of centrifuges known in the art, including but not limited to, tubular bowl, batch disc, nozzle disc, valve disc, open bowl, imperforate basket, and scroll discharge decanter types, can be used. Filtration by rotary vacuum drum or chamber filter can be used for concentrating fairly large microalgae. Flocculation is the collection of algal cells into an aggregate mass by addition of polymers, and is typically induced by a pH change or the use of cationic polymers. Foam fractionation relies on bubbles in the culture media which carries the algae to the surface where foam is formed due to the ionic properties of water, air and matter dissolved or suspended in the culture media.

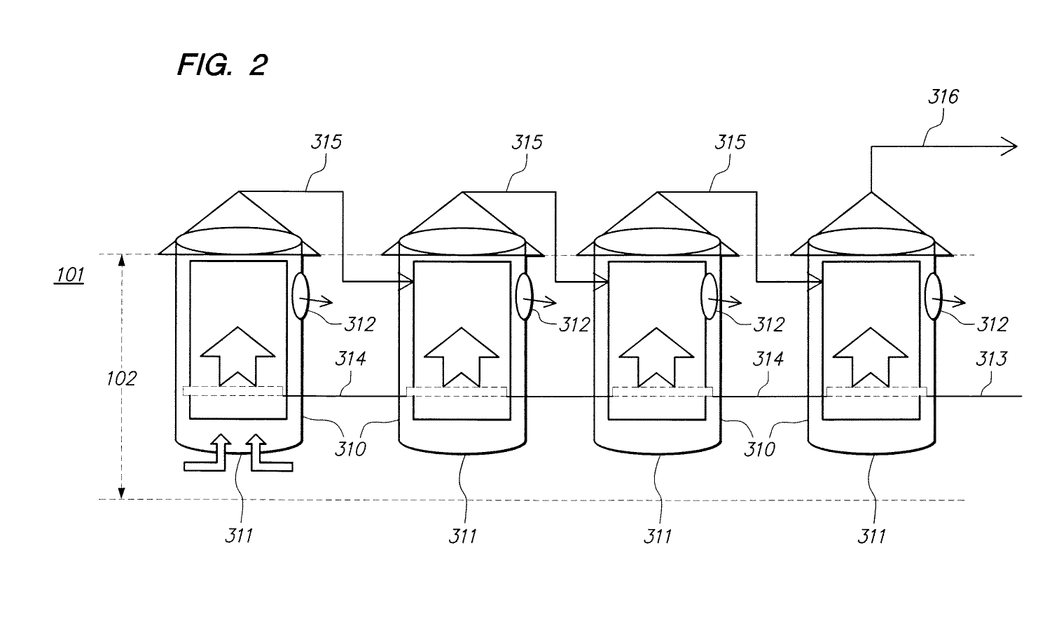

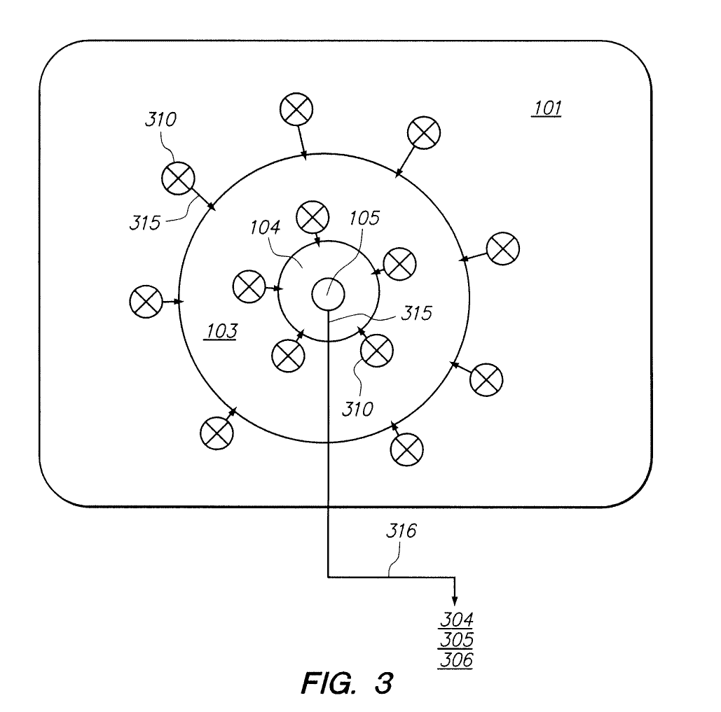

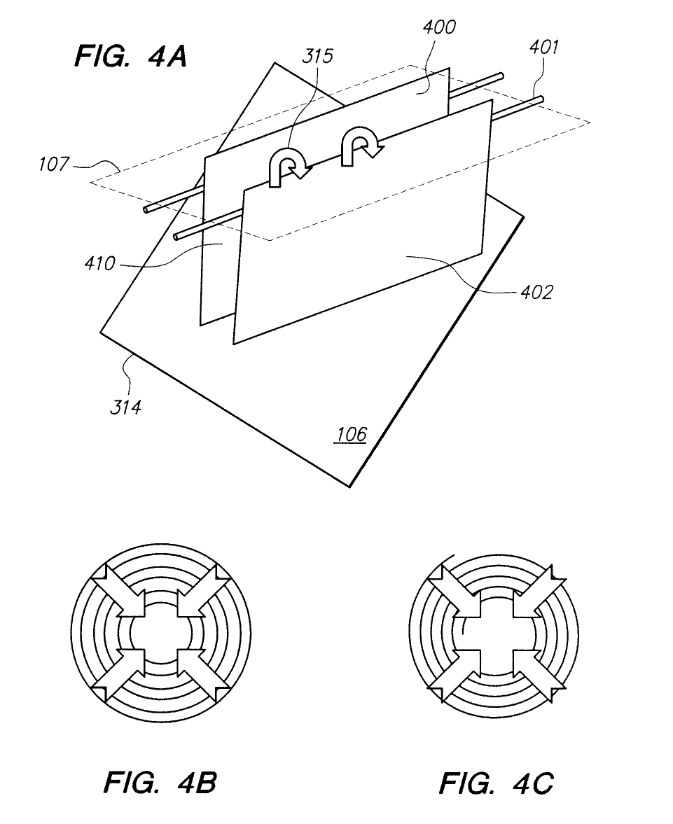

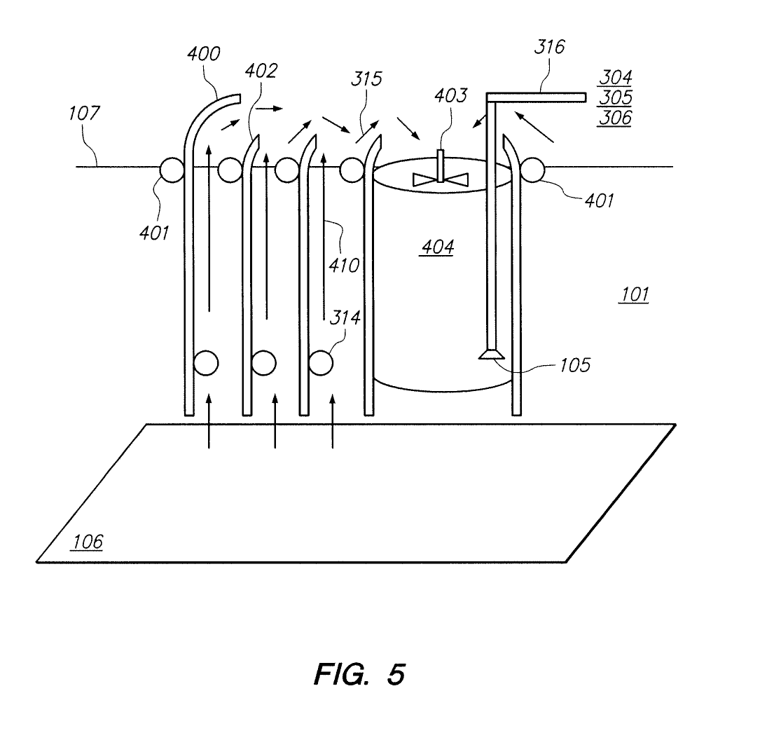

In one embodiment of the invention, the methods comprise using foam fractionation to concentrate the algae in at least one concentration step. In another embodiment, the invention provides a system comprising one or more foam fractionation means that can be used in a growth enclosure. The foam fractionation means can be connected serially so that the foam fraction from one unit is introduced or flows into another unit for a second round of foam fractionation. A foam fractionation means of the invention comprises a bubble-forming means to be placed in the water, and a means to separate at the top of a water column the foam fraction from the water. Bubbles in the culture media are formed by injecting gas, such as air, using a jet nozzle, sparger or diffuser, or by injecting water with bubbles using a venturi injector. The bubbles travel upwards within a water column and form a layer of foam comprising the algae at the top where the foam is removed from the surface. The foam fraction can be collected by any means, including but not limited to, mechanical or fluidic means, for example, by suction, siphoning, skimming, trapping, or by overflowing into an adjoining chamber. The foam condenses to form a concentrated algal composition. Examples of designs of foam fractionation means are provided in FIGS. 2 to 6.

Since the methods of the invention are provided for the production of biofuel, the lipid content is measured at one or more stages during the culture process, especially when the algae is concentrated or after the algae has been subjected to stress. Any methods known in the art can be applied. Depending on the yield, the algae may be cultured for an extended period of time, or the algae culture may be subjected to further stress, before harvesting. Any known technique can be applied to harvest and dewater the algae, see, for example, Fox, J. M., 1983, Intensive algal culture techniques. In: CRC Handbook of Mariculture Volume 1. McVey J P (ed) CRC Press, Florida, pp. 43-69 and Barnabe G., 1990, Harvesting micro-algae In: Aquaculture, Volume 1, Barnabe G. (ed.) Ellis Horwood, New York, pp. 207-212.

### 5.4 Lipids and Biofuel

The invention provides a biofuel, a biodiesel, or a biofuel feedstock comprising lipids derived from algal oil. Lipids produced by methods of the invention can be subdivided according to polarity: neutral lipids and polar lipids. The major neutral lipids are triglycerides, and free saturated and unsaturated fatty acids. The major polar lipids are acyl lipids, such as glycolipids and phospholipids. A composition comprising lipids and hydrocarbons of the invention can be described and distinguished by the types and relative amounts of key fatty acids and/or hydrocarbons present in the composition.

Fatty acids are identified herein by a first number that indicates the number of carbon atoms, and a second number that is the number of double bonds, with the option of indicating the position of the double bonds in parenthesis. The carboxylic group is carbon atom 1 and the position of the double bond is specified by the lower numbered carbon atom. For example, linoleic acid can be identified by 18:2 (9, 12).

Algae produce mostly even-numbered straight chain saturated fatty acids (e.g., 12:0, 14:0, 16:0, 18:0, 20:0 and 22:0) with smaller amounts of odd-numbered acids (e.g., 13:0, 15:0, 17:0, 19:0, and 21:0), and some branched chain (iso- and anteiso-) fatty acids. A great variety of unsaturated or polyunsaturated fatty acids are produced by algae, mostly with C₁₂ to C₂₂ carbon chains and 1 to 6 double bonds, mainly in cis configurations. Without limitation, it is contemplated that fatty acids isolated from the algae culture and of the invention comprise one or more of the following fatty acids: 12:0, 14:0, 14:1, 15:0, 16:0, 16:1, 16:2, 16:3, 16:4, 17:0, 18:0, 18:1, 18:2, 18:3, 18:4, 19:0, 20:0, 20:1, 20:2, 20:3, 20:4, 20:5, 22:0, 22:5, 22:6, and 28:1 and in particular, 18:1(9), 18:2(9,12), 18:3(6, 9, 12), 18:3(9, 12, 15), 18:4(6, 9, 12, 15), 18:5(3, 6, 9, 12, 15), 20:3(8, 11, 14), 20:4(5, 8, 11, 14), 20:5(5, 8, 11, 14, 17), 20:5(4, 7, 10, 13, 16), 20:5(7, 10, 13, 16, 19), 22:5(7, 10, 13, 16, 19), 22:6(4, 7, 10, 13, 16, 19).

The hydrocarbons present in algae are mostly straight chain alkanes and alkenes, and may include paraffins and the like having up to 36 carbon atoms. The hydrocarbons are identified by the same system of naming carbon atoms and double bonds as described above for fatty acids. Non-limiting examples of the hydrocarbons are 8:0, 9,0, 10:0, 11:0, 12:0, 13:0, 14:0, 15:0, 15:1, 15:2, 17:0, 18:0, 19:0, 20:0, 21:0, 21:6, 23:0, 24:0, 27:0, 27:2(1, 18), 29:0, 29:2(1, 20), 31:2(1,22), 34:1, and 36:0.

Examples of systems and methods for processing (or polishing) lipids such as algal oil into a biofuel feedstock or biofuel, can be found in the following patent publications, the entire contents of each of which are incorporated by reference herein: U.S Patent Publication No. 2007/0010682, entitled “Process for the Manufacture of Diesel Range Hydrocarbons;” U.S. Patent Publication No. 2007/0131579, entitled “Process for Producing a Saturated Hydrocarbon Component;” U.S. Patent Publication No. 2007/0135316, entitled “Process for Producing a Saturated Hydrocarbon Component;” U.S. Patent Publication No. 2007/0135663, entitled “Base Oil;” U.S. Patent Publication No. 2007/0135666, entitled “Process for Producing a Branched Hydrocarbon Component;” U.S. Patent Publication No. 2007/0135669, entitled “Process for Producing a Hydrocarbon Component;” and U.S. Patent Publication No. 2007/0299291, entitled “Process for the Manufacture of Base Oil.” Products of the invention made by the processing of algae-derived biofuel feedstocks can be incorporated or used in a variety of liquid fuels including but not limited to, diesel, biodiesel, kerosene, jet-fuel, gasoline, JP-1, JP-4, JP-5, JP-6, JP-7, JP-8, Jet Propellant Thermally Stable (JPTS), Fischer-Tropsch liquids, alcohol-based fuels, including ethanol-containing transportation fuels, and other biomass-based liquid fuels, including cellulosic biomass-based transportation fuels and algae pyrolysis-derived oils.

The present invention may be better understood by reference to the following non-limiting examples, which are provided only as exemplary of the invention. The following examples are presented to more fully illustrate the preferred embodiments of the invention. The examples should in no way be construed, however, as limiting the broader scope of the invention.

### 6. EXAMPLES OF SYSTEMS OF THE INVENTION

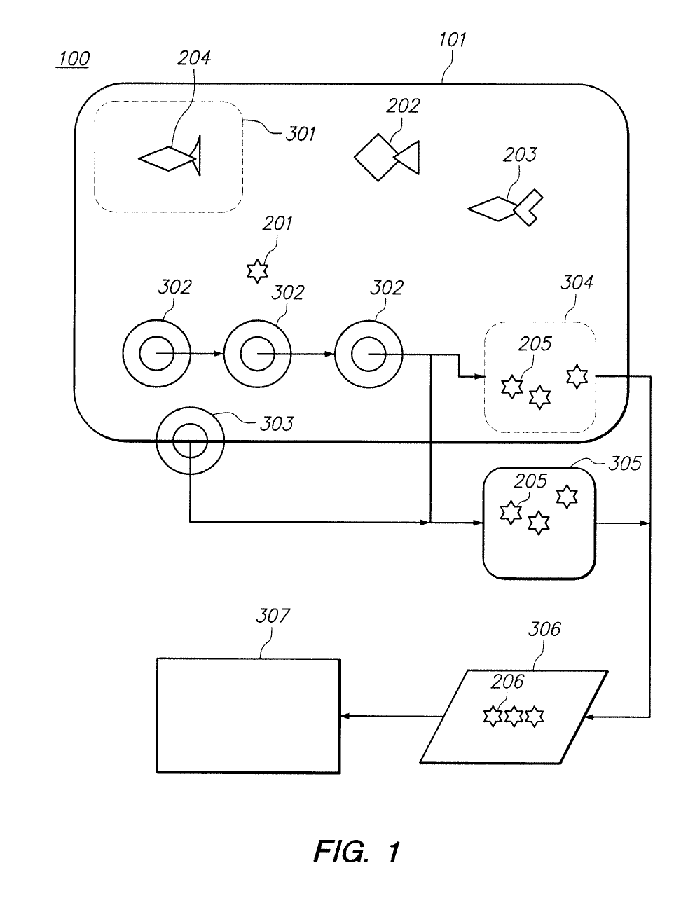

An overview of a method 100 of obtaining biofuel from fish, according to some embodiments of the invention, is described below and in FIG. 1. Referring to FIG. 1, first, an environment, an aquatic enclosure, a species of fish and a species of algae are selected to enhance energy production from the system 110. The environment and type of aquatic enclosure to be established in that environment are selected to be hospitable to growth of the species of fish and algae. The environment is selected to be non-arable land, so as to avoid using land that could otherwise be used for food crops. The selected type of aquatic enclosure is then established in the selected environment 120.

A plurality of fish of the selected species and an algae composition comprising the selected species of algae are then introduced into the fish enclosure 130. The size of the populations is selected based, in part, on the size and characteristics of the enclosure and the growth characteristics of the particular species. The plurality of algae can be exposed to light from the sun 140, which enables growth of the algae. A majority portion of the algae is harvested with the population of fish 150. Usefully, the portion of algae that is not consumed can reproduce in the enclosure and thus replenish the algae population. In certain embodiments, an equilibrium may be sustained between the fish population and the algae that continue to grow in the fish enclosure.

After a predefined amount of time (e.g., after the fish grow to a specified size, or after the growth rate of the fish drop below a specified value), a plurality of fish are gathered 150, e.g., using conventional fishery techniques such as netting. Optionally, some fish are left in the enclosure to reproduce and thus replenish the fish population. In other embodiments, substantially all of the fish are gathered and processed for biofuel (170). According to the invention, a new batch of fish of the selected species is introduced into the enclosure. The cycle of adding algae followed by algal growth (140), harvesting the algae (150), gathering the fish (160), conversion of the fish into biofuel (170), and introduction of a new batch of fish can be repeated as many times as desired, so long as the environment and aquatic enclosure remain suitable for growth of the fish population.

In another embodiment of the invention, the fishes and algae are grown separately from each other for at least part of the time before the fishes are allowed to harvest the algae. FIG. 2 illustrates a system 200 that grows the algae separately from the fishes. System 200 includes an algae enclosure 210, a fish enclosure 220, a gate 230, and an aquatic passageway 240 for transferring algae from algae enclosure 210 to fish enclosure 220 when gate 230 is opened. Selected species of algae are introduced into the water in algae enclosure 210, which is connected to CO₂ source 250 and/or nutrient source 260. Because there are substantially no fish in algae enclosure 210, the growth of algae 211 is essentially unchecked. Then, after the algae 211 reaches a sufficient density, the gate 330 is opened and the algae flows through aquatic passageway 340 into fish enclosure 320. There, fishes 221 harvest algae 222 and grow to a desirable size or weight. After the period of growth, the fishes are gathered or harvested by device 270 and move by a conveyor 280 to fish processing plant 300 where the fish lipids are extracted. The fish lipids can be upgraded into biofuel in reactor 400.

### 7. PILOT SCALE ALGAE CULTURE

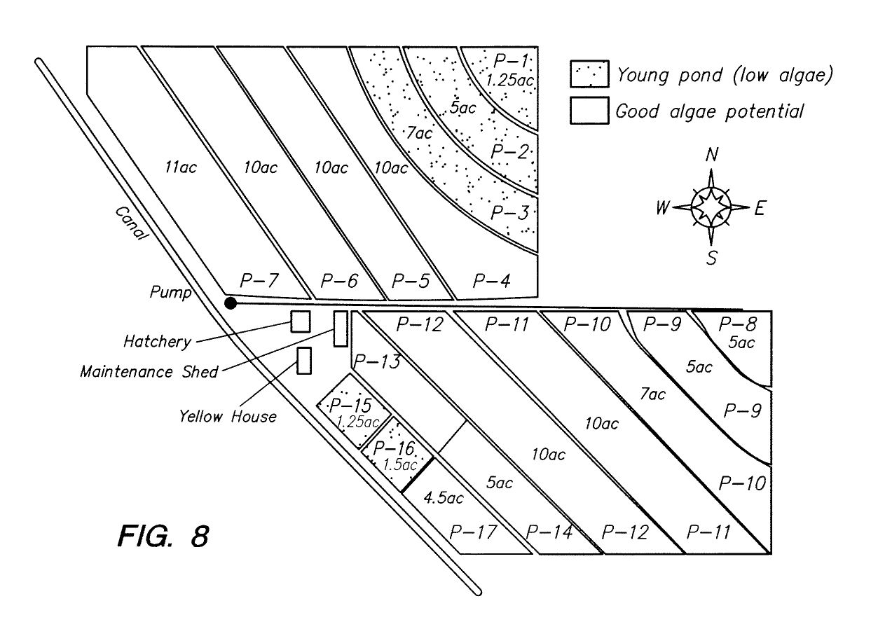

A series of pilot scale studies was carried out to study the culturing of algae in the open ponds of a fish farm and harvesting of the algae by mechanical means. See FIG. 8 for a map of the fish farm. The results demonstrate that a biofuel feedstock (lipids) can be produced from algae harvested from an outdoor open continuous culturing system that comprises fish.

### 7.1 Preliminary Analysis of Algal Biomass

The objective of the following study is to assess qualitative and quantitative features of 17 ponds in the fish farm that can affect algae growth. The nitrate level, nitrite level, pH, KH (carbonate hardness), GH (general hardness), water temperature of 17 ponds were recorded. Carbonate hardness is a measure of carbonate and bicarbonate ions, and used as to estimate carbon dioxide reserves in the water. General hardness measures the magnesium and calcium ion concentrations in the water. The color and timing of appearance of algal mats and cyanobacteria (“cyano”) in the ponds were also recorded. The same measurements are made periodically to observe how these features of the ponds change as the weather changes from winter to summer. Table 1 shows the data collected from an area of each pond (as indicated by direction) between 9 am and 11 am on a sunny day in December 2007. Ambient air temperature was 52° F. to 60° F.

TABLE 1

| | | | | | KH | | |
|---|---|---|---|---|---|---|---|
| | | | | | (carbonate = | | |
| POND # | Color | Nitrate | Nitrite | pH | stored CO₂) | GH | Observations |
| 1 NE | Mixed green + | 20 | 0.5 | 9.0 | 240 | 180 | Very small pond |
| | cyano | | | | | | |
| 2 NE | Bright green | 20 | 0 | 9 | 240 | 180 | Waspy under layer |
| 3 NE | +mat, +cyano | 20-40 | 0.5 | 9 | 240 | 180 | Definitely still cyano |
| 4 NE | +mat | 20 | 0 | 9 | 240 | 180 | Green “skin-like” mat in |
| | | | | | | | NE center |
| 5 NE | NE +mat | 20-40 | 0.5 | 9 | 240 | 180 | Green “skin-like” mat in |
| | | | | | | | NE corner |
| 6 NE | Green, | 20 | 0 | 9 | 240 | 180 | Took sample from just |
| | floating mat | | | | | | below surface mat |
| 7 NE | Green/brown | 0-20 | 0 | 8.5-9 | 240 | 180 | Very green, can't tell if |
| | | | | | | | cyano or algae b/c no |
| | | | | | | | mats accumulated |
| 8 NE | clear | 0 | 0 | 7.5 | 120 | 180 | |
| 9 NW | Green/red | 20 | 0 | 9 | 240 | 180 | Calm, no mats |
| | color | | | | | | |
| 10 NW | Turning | 20 | 0.5 | 9 | 240 | 180 | No mats, brown-green |
| | green/brown | | | | | | in color |
| 11 SE | Very brown | 20 | 0 | 9 | 240 | 180 | Brown water, green |
| | | | | | | | mat accumulated in SE |
| | | | | | | | corner |
| 12 SE | Green/brown | 0-20 | 0 | 9 | 240 | 180 | Still green, turning |
| | | | | | | | brown/red in color. |
| | | | | | | | Mats accumulating |
| | | | | | | | weakly in SE corner |
| 13 SE | Very bright | 0-20 | 0 | 9 | 240 | 180 | Algae true green, no |
| | green | | | | | | mats, no red/brown |
| | | | | | | | tinge to water |
| 14 SE | Green/cyano+ | 40-80 | 3 | 9 | 240 | 180 | Weak blue/green mats |
| | | | | | | | in SE corner |
| 15 NW | Calm, cyano | 0 | 0 | 9 | 240 | 180 | No mats |
| | green | | | | | | |
| 16 NW | brown | 0-20 | 0 | 9 | 240 | 180 | Took sample from mid- |
| | | | | | | | NW, calm, no mats |
| 17 NW | Cyano green | 0 | 0 | 9 | 240 | 180 | Minor cyano mat in SW |
| | | | | | | | corner |

The biomass in three batches of pond water from two ponds, each about 200 gallons, were harvested and analyzed. The results are shown in Table 2 below. Pond water was collected from areas where the algae appeared to be accumulating on the surface of the ponds. This was largely affected by wind direction and speed. The algae tended to be pushed by the wind into quiescent corners of the ponds. A series of flexible hoses were connected using quick-connect fittings (depending on the length required) to the inlet side of a pump. A piece of screening on the end of the inlet hose kept out large pieces of dirt, grass or small fish. The inlet was attached to a pole which is used to place the hose at or below the surface of the algae mats. Collected pond water was centrifuged by a simple decanting type of centrifuge (US Centrifuge, model M212).

TABLE 2
Physical characteristics of harvested biomass.

| | | Paste Net | Total Feed | Feed % | |
|---|---|---|---|---|---|
| Run# | Pond # | Weight | Volume | Solids | Paste % Solids |
| 1 | 11 | 347 g | ~200 gal | 0.18 | 18.4 |
| 2 | 5 | 406 g | ~200 gal | 0.17 | 15.0 |
| 3 | 11 | 420 g | ~200 gal | 0.31 | 19.0 |

Diluted water samples from the centrifuge bowl eluents were examined by light microscope. Table 3 shows the observations from three runs.

TABLE 3
Observations of diluted water samples by microscope.

| Run | Diatoms | Chlorophyceae | Trachelamonas | Cyanobacteria |
|---|---|---|---|---|
| 1, Pond 11 | 30-40% per cell | Up to 15% per cell | Very few | At least 50%, per |
| | counting | counting | | cell counting |
| 2, Pond 5 | Less than 10% | ~10-15% | None observed | At least 50%, per |
| | | | | cell counting |
| 3, Pond 11 | 20% per cell | ~15% | More, but still less | At least 50%, per |
| | counting | | than 5% | cell counting |

### 7.2 Harvesting Algae by Centrifugation

The following study was designed to investigate a process for harvesting algae from pond water, the yield of algae, and extraction of lipids from the harvested algae.

A total of 28 batches of pond water were processed. The average batch size was 804 liters (212 gallons). The solids concentrations of the collected pond water were measured—two types of solids in the pond water, i.e., total solids and suspended solids. Total solids was based on initial and final weights on a moisture balance:

% TS=[WeightFinal/WeightInitial]×100

Moisture balances operate on the simple principle that all moisture in the sample is removed by evaporation once the weight of the sample has stopped changing after heating in a vented chamber. It therefore captures all the solids in the sample, including both dissolved and suspended solids. Suspended solids measurement is based on passing of the samples through a sub-micron filter and measuring the dry weight of material captured per unit volume filtered. The results show that the total solids in the pond water are actually around ten-fold higher than the suspended solids in the collected pond water. Most of the solids found in the pond water came from dissolved solids present in the water that was not algal biomass. Indeed, the extraordinarily high dissolved solids in the water may reflect the extremely poor quality and high salinity of the local river which drained into the pond. On average, the pond water fed to the centrifuges contained 2 to 4 (average=2.65) grams per liter of total solids, of which only 0.31 grams per liter was actually suspended solids that were captured in the centrifuge.

Table 4 is a summary of the data gathered in this experiment. “U” and “A” in the batch numbers refer to the type of centrifuge used (see below). Total solids includes both dissolved and suspended solids. Concentration factor is the ratio of solids concentration in the paste to the suspended solids concentration in the pond water feed. Averages and standard deviations exclude data from run U-7 because its mass closure was so poor.

TABLE 4

| | | | Feed | Feed | | Total | Total | | | |
|---|---|---|---|---|---|---|---|---|---|---|
| | | Feed | total | suspended | Paste | suspended | recovered | Solids | | |
| | | volume | solids | solids | solids | solids in feed | solids in | recovery | Mass | Conc. |
| ID | Date | (liters) | (g/l) | (g/l) | (g/l) | (kg) | paste (kg) | efficiency | Closure | Factor |
| U-1 | 3-Oct | | | | | | | | | |
| U-2 | 4-Oct | 284 | 2.68 | | 167 | | 0.310 | | | |
| U-3 | 9-Oct | 719 | | | | | | | | |
| U-4 | 10-Oct | 809 | 2.78 | | 156 | | 0.275 | | | |
| U-5 | 12-Oct | 878 | | | 160 | | 0.106 | | | |
| U-6 | 12-Oct | 845 | 2.85 | 0.256 | 170 | 0.217 | 0.137 | 63% | 72% | 664 |
| U-7 | 16-Oct | 928 | 2.79 | 0.243 | 154 | 0.226 | 0.710 | 314% | 324% | 633 |
| U-8 | 17-Oct | 796 | 3.81 | 0.642 | 148 | 0.511 | 0.143 | 28% | 33% | 230 |
| U-9 | 25-Oct | 813 | 3.20 | 0.354 | 174 | 0.288 | 0.262 | 91% | 108% | 492 |
| U-10 | 26-Oct | 815 | 2.70 | 0.265 | 169 | 0.216 | 0.159 | 73% | 99% | 638 |
| U-11 | 30-Oct | 825 | 2.75 | 0.383 | 170 | 0.316 | | | | 443 |
| U-12 | 1-Nov | | | | | | | | | |
| A-1 | 2-Nov | | | | | | | | | |
| A-2 | 2-Nov | 970 | | 0.284 | 0 | 0.275 | | | | |
| A-3 | 6-Nov | 804 | | 0.200 | 97 | 0.161 | | | | 484 |
| A-4 | 6-Nov | 823 | | 0.223 | 107 | 0.183 | | | | 480 |
| A-5 | 7-Nov | 804 | | 0.285 | 89 | 0.229 | 0.108 | 47% | 50% | 313 |
| A-6 | 7-Nov | 800 | 1.63 | 0.350 | 142 | 0.280 | 0.159 | 57% | 60% | 405 |
| A-7 | 8-Nov | 807 | 2.88 | 0.512 | 83 | 0.414 | 0.292 | 70% | 75% | 162 |
| A-8 | 9-Nov | 789 | 2.57 | 0.192 | 118 | 0.152 | 0.173 | 114% | 123% | 615 |
| A-9 | 9-Nov | 839 | 3.47 | 0.284 | 107 | 0.238 | 0.177 | 75% | 80% | 378 |
| A-10 | 13-Nov | 827 | 3.21 | 0.206 | 45 | 0.171 | 0.141 | 83% | 86% | 219 |
| A-11 | 13-Nov | 822 | 1.94 | 0.000 | 104 | 0.000 | 0.151 | | | |
| A-12 | 14-Nov | 798 | 2.24 | 0.374 | 100 | 0.298 | 0.153 | 51% | 58% | 267 |
| A-13 | 14-Nov | 837 | 2.56 | 0.440 | | 0.368 | | | | |
| A-14 | 15-Nov | 825 | 0.00 | 0.329 | 124 | 0.271 | 0.165 | 61% | 67% | 378 |
| A-15 | 15-Nov | 787 | 3.28 | 0.383 | 127 | 0.301 | 0.159 | 53% | 57% | 332 |
| A-16 | 16-Nov | 857 | 3.12 | 0.233 | 94 | 0.199 | 0.153 | 77% | 85% | 404 |
| | Total | 20,102 | | | | 5.314 | 3.932 | | | |
| | Avg | 804 | 2.65 | 0.307 | | 0.253 | 0.207 | | | 419 |
| | StDev | 120 | 0.84 | 0.134 | | 0.106 | 0.139 | | | 152 |

Two centrifuges were used to process over 20,000 liters of pond water. Pond water was collected as described in Section 7.1, stored on a truck tank and transferred to a feed tank. A compressed air-driven diaphragm pump was used to draw liquid from the outlet at the bottom of the feed tank to the inlet of the centrifuge.

The first centrifuge tested was a decanting centrifuge from US Centrifuge (Model M212, see “U” batch numbers). The unit spun an open bowl or basket at speeds of around 1,500 RPM. Solids-containing feed was pumped into the top of the unit. The liquid was forced to the bottom of the spinning bowl. Centrifugal force pushes the solids against the vertical walls of the centrifuge. As long as the flow into the bowl was kept low enough, solids could be captured on the side wall, even as liquid flows up through the bowl. The clear liquid was decanted by forcing it to flow in an annular space surrounding the spinning bowl. A large opening in the side wall was used to collect the clarified liquid (referred to as centrate) in an open container. A removable liner in the bowl allows drainage of residual liquid and collection of the remaining solids. The centrifuge was also run without additional input liquid for 15 minutes to remove additional solids.

A second centrifuge tested was a high speed disk stack centrifuge from Alfa Laval (see “A” batch numbers”) which was designed for very high removal rates of solids of particles sizes as low as 0.5 to 1.0 microns in diameter. Its ability to recover smaller particles sizes was related to its higher speed of rotation and a set of disk stacks which created a tremendous amount of area for settling of solids as liquid travels up the space between the disks. This centrifuge continuously discharged solids without interruption but required the use of water to flush the solids out leading to dilution. Average flow through the unit was typically around 12 liters per minute, three times the flow rate achieved with the decanting type centrifuge. The relatively low speed decanting centrifuge (US Centrifuge) achieved an average increase in solids concentration of 517-fold relative to the incoming pond water feed. The high speed disk stack centrifuge (Alfa Laval) achieved an increase in solids concentration of 370-fold.

While solids removal efficiency was very high for both centrifuges (about 94%), the average calculated solids recovery efficiency—defined as the ratio of the total solids captured in the solids coming out of the centrifuge to the total suspended solids present in the feed—was about 61%. The efficiency for run U-7 was excluded from this chart because it showed an erroneous recovery efficiency of over 300%. Despite variability in yield and mass closure (about 75%), all of the runs were able to recover 0.17 grams of solids per liter of pond water processed.

A total of 35 kilograms of an algal composition—an algal paste (concentrated solids) was obtained. Recovered solids contained in the paste weighed around 4 kilograms in total (9 lbs) on a dry basis. The composition of the solids was subjected to the following standard assays: total solids analysis which measures moisture content within sample with numbers corrected on a dry weight basis; ash determination assay which measures the amount of inorganic material present structurally and non-structurally as extractables; exhaustive ethanol/water extractives which remove non-structural material from the biomass sample to prevent interferences during a number of assay including free sugar determination; carbohydrates analysis that determines glucose, xylose, galactose, arabinose and mannose concentrations in the sample as a measure of cellulose and hemicellulose concentrations in the biomass; amylase enzyme assay which determines starch content, protein content assay based on LECO combustion methods; bomb calorimetry to determine the sample's BTU content; and lipid analysis which measures total extractable lipids, carbon chain length, C4-C24 fatty acids, saturated, unsaturated, polyunsaturated, and mono fatty acids. An acid hydrolysis/ether extraction analysis for the lipids was also performed. This assay identifies all fatty acids present in the biomass, including those that are present in the cell membrane and in lipid pigments. An ether extraction alone will only capture those lipids that are present as storage lipids (neutral lipids or triglycerides). This assay involved the incubation of the sample in a known concentration of HCl solution with ethanol for one hour. The sample was then run through an ether extraction. The ether extracts are collected and dried to get a gravimetric value. Table 5 shows the composition of the solids (polar lipids=difference between total lipids and neutral and include unknown components that did not show up as C4 to C24 compounds).

TABLE 5
Relative amounts of materials in the solids

| Composition | Percent |
|---|---|
| Moisture | 4.59% |
| Ash | 7.27% |
| Protein | 55.98% |
| Water extractives | 17.52% |
| Polar lipids | 7.04% |
| Neutral lipids | 0.09% |
| Glucan | 7.47% |
| Galactan | 3.5% |
| Mannan | 1.07% |
| Total | 99.94% |

By far the largest component is protein, representing more than half of the total weight in the solids. Sugars—comprising 12% of the total solids—include polymers of glucose, galactose and mannose. Essentially no storage lipids (triglycerides or neutral lipids extracted in ether) are present.

Total lipids (captured in the acid hydrolysis/ether extraction) are around 7% of the total weight of dry solids. The following Tables 6, 7, and 8 show the fatty acid chains identified in the acid hydrolysis/ether extract. Prep A and Prep B refer to replicate analyses. The nomenclature in front of each fatty acid chain name refers to the number of carbons in the chain and the number unsaturated bonds in the chain. The numbers in the parentheses following the fatty acid chain name indicate the type of unsaturated bond (cis versus trans) and the carbon number (location) of each unsaturated bond.

TABLE 6
Profile of Saturated Fatty Acids

| | | | Ave |
|---|---|---|---|
| | Fatty Acids (g/100 g) | | Concentration |
| Saturated Fatty Acids | Prep A | Prep B | (g/100 g) |
| C4:0 butyric | 0 | 0 | 0 |
| C6:0 hexanoic | 0 | 0 | 0 |
| C8:0 octanoic | 0 | 0 | 0 |
| C10:0 decanoic | 0 | 0 | 0 |
| C12:0 lauric | 0 | 0 | 0 |
| C13:0 tridecanoic | 0 | 0 | 0 |
| C14:0 myristic | 0 | 0 | 0 |
| C15:0 pentadecanoic | 0 | 0 | 0 |
| C16:0 palmitic | 1.43 | 1.77 | 1.60 |
| C17:0 heptadecanoic | 0 | 0 | 0 |
| C18:0 stearic | 0.068 | 0.091 | 0.079 |
| C20:0 arachidic | 0 | 0 | 0 |
| C21:0 heneicosanoic | 0 | 0 | 0 |
| C22:0 behenic | 0 | 0 | 0 |
| C23:0 tricosanoic | 0 | 0 | 0 |
| C24:0 lignoceric | 0 | 0 | 0 |
| Totals | 1.50 | 1.86 | 1.68 |

TABLE 7
Profile of Monosaturated Fatty Acids

| | | | Average |
|---|---|---|---|
| | Fatty Acids (g/100 g) | | Concentration |
| Monounsaturated Fatty Acids | Prep A | Prep B | (g/100 g) |
| C14:1 myristoleic (cis-9) | 0 | 0 | 0 |
| C15:1 pentadecinoic (cis-10) | 0 | 0 | 0 |
| C16:1 palmitoleic (cis-9) | 0.448 | 0.531 | 0.490 |
| C17:1 heptadecenoate (cis-10) | 0 | 0 | 0 |
| C18:1 oleic (cis-9) | 0.148 | 0.189 | 0.1686 |
| C20:1 eicosenoic (cis-11) | 0 | 0 | 0 |
| C22:1 erucic (cis-13) | 0 | 0 | 0 |
| C24:1 nervonic (cis-15) | 0 | 0 | 0 |
| Totals | 0.596 | 0.721 | 0.658 |

TABLE 8
Profile of Polysaturated Fatty Acids.

| | | | Average |
|---|---|---|---|
| | Fatty Acids (g/100 g) | | Concentration |
| Polyunsaturated Chains | Prep A | Prep B | (g/100 g) |
| C18:2 linoleic (cis-9,12) | 0.503 | 0.620 | 0.561 |
| C18:3 y-linolenic (cis-6,9,12) | 0.200 | 0.239 | 0.219 |
| C18:3 linolenic (cis-9,12,15) | 0.610 | 0.727 | 0.668 |
| C20:2 eicosadienoic (cis-11,14) | 0 | 0 | 0 |
| C20:3 eicosatrienoic (cis- | 0 | 0 | 0 |
| 8,11,14) | | | |
| C20:3 eicosatrienoic (cis- | 0 | 0 | 0 |
| 11,14,17) | | | |
| C20:4 arachidonic (cis- | 0 | 0 | 0 |
| 5,8,11,14) | | | |
| C20:5 eicosapentanoic (cis- | 0 | 0 | 0 |
| 5,8,11,14,17) | | | |
| C22:2 docosadienoic (cis-13,16) | 0 | 0 | 0 |
| C22:6 docosahexaenoic (cis- | 0 | 0 | 0 |
| 4,7,10,13,16,19) | | | |
| Totals | 1.31 | 1.59 | 1.45 |

The lipids in the algal biomass were mostly C-16 and C-18 chains. FIG. 12 shows the distribution of fatty acid chains relative to the distribution of fatty acid chains for palm oil (a feedstock known to work well for renewable diesel and jet fuel production). While the lipids extracted from the algal biomass and palm oil have in common a large C16 peak (palmitic and palmitoleic acid), the lipids also have a very substantial amount of polyunsaturated C18 fatty acids. The neutral lipids found in the ether extract have a similar distribution, though with greater proportions of unsaturated fatty acids.

### 7.3 Lipid Extraction Methods

Four different solvent extraction methods for extracting lipids from a biomass were tested: (1) Bligh Dyer (Salt)—chloroform, methanol; (2) Soxhlet—either hexane or ethanol; (3) Nichols—isopropanol, chloroform; and (4) Hara—hexane, isopropanol. Based on ease of technique, time, solvent amounts, percent lipid recovery, and reproducibility, two extraction procedures are preferred, namely the Bligh Dyer Salt method and the Soxhlet method.

The Bligh Dyer Salt technique uses three solvents chloroform, methanol, and salt water (NaCl) to extract both polar and non-polar lipids from a wet sample of algae. The chloroform pulls out non-polar and polar lipids, while the methanol extracts polar lipids. The salt water helps to partition more of the lipids into the chloroform layer. This extraction process requires vortexing (mixing) and centrifugation. After the centrifugation, three visible layers are formed. The top yellowish layer is a methanol, water mixture layer and may contain salts, proteins, sugars. The middle layer is mainly water and may contain proteins and sugars. The bottom layer (chloroform layer) contains the lipids. Average lipid recovery of 20% was obtained by this technique.

Another technique used a Soxhlet extraction apparatus and hexane as the extracting solvent. During this extraction, a dry algae sample was used and placed in an extraction thimble. This setup allowed the hexane to drip onto the algae sample. As the solvent dripped, the non-polar lipids were extracted. This extraction process was repeated over twenty-four hours and yielded an average lipid recovery of 10%.

### 7.4 Drying

The drying of recovered solids was carried out using a plow mixer/dryer (Littleford-Day model M-5-R). The algae paste was added periodically throughout the test as the volume in the chamber was reduced by drying. The unit was heated with low pressure steam (˜225° F.) while under vacuum. The material did dry to a final moisture of approximately 6.5% after a ten (10) hour cycle. The M-5-R had processed a total 10,335 grams of algae paste/slurry. Approximately 0.775 kilograms of dry material were recovered from the reactor, making the yield of dry material 7.5% (kg/kg).

### 7.5 Flocculation and Dissolved Air Flotation (DAF)

A bench-scale DAF tests using a batch DAF unit which consists of an air saturation tank and a flotation tank was conducted. Samples of pond water were first treated with a chemical coagulant, flocculated, settled and decanted to produce a simulated recycle water for the air saturation tank. More pond water was then treated with the coagulant, flocculated and transferred to the flotation tank. After the simulated recycle water was saturated with air under pressure, it was released into the flocculated water in the flotation tank at atmospheric pressure. Bubbles formed and carried were solids to the surface. Samples of the raw water, simulated recycle and subnatant were collected and analyzed for total suspended solids (TSS). The DAF float was also analyzed for total solids. Chemical coagulants used were provided by the following manufacturers: (1) Monolyte 5070 v. 5 as used by a municipal authority to coagulate and flocculate algae before removal by DAF. Monolyte 5070 v. 7, a newer version, was used; (2) HaloSource product, Storm Klear, a formulation of chitosan which has been used at construction sites to coagulate sediment from storm water prior to discharge. See Table 9 for results.

The best percent recovery of algae was obtained using Monolyte 5070 v. 7 at the lowest dose tested (4 ppm) and the lowest recycle ratio tested (25 percent). The DAF subnatant contained 67 mg/L, which was just slightly less than the subnatant TSS (71 mg/L) using the same chemical with a 50 percent recycle ratio. The percent removals were different because the lower recycle ratio resulted in a higher initial TSS concentration, since the pond water was being diluted less. The flocculated solids formed a DAF float very quickly—within seconds. The float concentrations were all in the range of 5 percent to 6 percent solids. Since the initial solids concentrations were approximately 150 mg/L, the concentration factor in the DAF was approximately 300 to 400.

TABLE 9

| | | | | | | Float |
|---|---|---|---|---|---|---|
| | Dose | Recycle | Initial TSS | Final TSS | Percent | Solids |
| Chemical | (ppm) | Ratio | (mg/l) | (mg/l) | Recovery | Present |
| None | — | 50 | 139 | 150 | 0 | No float |
| Monolyte 5070 v. 7 | 4 | 50 | 139 | 71 | 49 | 6.3 |
| Monolyte 5070 v. 7 | 4 | 25 | 157 | 67 | 57 | 5.4 |
| Monolyte 5070 v. 7 | 5 | 50 | 155 | 99 | 36 | na |
| Monolyte 5070 v. 7 | 10 | 50 | 145 | 79 | 46 | 5.5 |
| Monolyte 5070 v. 7 + | 1, 30 | 50 | 139 | 122 | 12 | 5.6 |
| Storm Klear | | | | | | |
| (Chitosan) | | | | | | |

### 8. EFFECT OF STRESS ON LIPID LEVELS

The following experiment is designed to investigate nutrient limitation as a stressor on the lipid level of algae. The 9-day experiment allows nutrients in the water to be depleted by the biomass and drive the algae to lipid accumulation

Two 500-gallon tanks were seeded with water containing algal biomass obtained from West Pond 4 (W4). Samples of biomass were collected at five time points for analysis. Samples were taken at T=0, T=2 days, T=6 days, T=8 days and T=9 days, providing five sampling points for each tank. The pH of the water in Tank B was adjusted once daily with CO₂ and the water in Tank C was the control with no CO₂ adjustment. The water in the tanks were sporadically mixed in the morning and evening.

When a sample of the biomass at T=0 was examined under light microscope, the presence of three dominant groups of algae was observed: (i) Cylindrotheca species, possibly C. closterium and/or C. setaceum; (ii) radial diatoms, possibly Thalassiasira species; and (iii) Euglena species, including large Euglena specimens and E. gracilis. Lipid analysis of the biomass was carried out by an acid-methanol procedure. A summary of the data is shown in Table 10 below.

TABLE 10

| TANK | % Solids | AM Temp | PM Temp | pH | Action | |
|---|---|---|---|---|---|---|
| B | 0.317 | | 86.1 | 7 | CONTROL SAMPLE + | T = 0 Wed |
| | | | | | CENT | |
| C | 0.467 | | 86.3 | 9 | | B |
| B | 0.284 | 73.3 | | 7 | | T = 1 d |
| | | | | | | Thurs |
| C | 0.452 | 73.8 | | 8 | | |
| B | 0.318 | 78.3 | 87.5 | 7 | CO2 SAMPLE + CENT | T = 2 d Fri |
| C | 0.48 | 80.4 | 87.3 | 8.5 | CONTROL SAMPLE + | |
| | | | | | CENT | |
| B | 0.369 | 71.9 | | 7 | | T = 3 d Sat |
| C | 0.534 | 72.7 | | 8.5 | | |
| B | 0.409 | 76.2 | | 7 | | T = 4 d Sun |
| C | 0.543 | 77.3 | | 8.5 | | |
| B | | | | | | T = 5 d Mon |
| C | | | | | | |
| B | 0.407 | 80.1 | 94.4 | 7 | CO2 SAMPLE + CENT | T = 6 d Tues |
| C | 0.48 | 85.3 | 93.6 | 8.5 | CONTROL SAMPLE + | |
| | | | | | CENT | |
| B | 0.389 | 75.3 | 93.8 | 7 | | T = 7 d Wed |
| C | 0.508 | 75.5 | 94.6 | 8.5 | | |
| B | 0.388 | 76.5 | 93.5 | 7 | CO2 SAMPLE + CENT | T = 8 d |
| | | | | | | Thurs |
| C | 0.466 | 82.7 | 94.1 | 8.5 | CONTROL SAMPLE + | |
| | | | | | CENT | |
| B | 0.462 | 68.1 | | 7 | CO2 SAMPLE + CENT | T = 9 d Fri |
| C | 0.467 | 71.1 | | 8.5 | CONTROL SAMPLE + | |
| | | | | | CENT | |

The data also has an outlier on Day 8 that is probably due to a process error.
The results show that the lipid contents of the algae in the W4 feed were increased from 5%/3% (crude/ID FA) to 11%/6% (crude/ID FA) at Day 6. The results demonstrate that the metabolism of lipids in algae can be driven in a particular direction by manipulating the growth environment. (ID FA is percentage of total fatty acids identified by gas chromatography/mass spectrometry.)

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

What is claimed:

1. A method for producing a biofuel feedstock comprising culturing algae in an aquatic composition that comprises a plurality of fish, wherein one or more conditions of the aquatic composition are controllably modified by the plurality of fish to promote algal growth, harvesting the algae from said aquatic composition, and extracting lipids from the algae, wherein the lipids are used as a biofuel feedstock.

2. The method of claim 1, further comprises providing a system wherein the algae and the plurality of fish are cultured in a body of water.

3. The method of claim 1, further comprising inducing the algae to accumulate lipids by a stressor

4. The method of claim 1, further comprising processing the lipids to form the biofuel feedstock.

5. The method of claim 1, wherein said one or more conditions of the aquatic composition controllably modified by the fish comprise at least one of nitrogen concentration, phosphorous concentration, carbon dioxide concentration, oxygen level, temperature uniformity, zooplankton level, mollusk population, and crustacean population.

6. The method of claim 2, wherein the plurality of fish is confined in one or more cages in the body of water, and wherein said one or more conditions of the aquatic composition is controlled by increasing or decreasing the degree of mixing in the body of water.

7. The method of claim 3, further comprising measuring the content of lipids in a sample of the algae, and repeating the culturing step and inducing step at least one time after the measuring step.

8. The method of claim 1, further comprising concentrating the algae to form an algal composition prior to harvesting the algae.

9. The method of claim 3, further comprising concentrating the algae prior to inducing the algae to accumulate lipids.

10. The method of claim 3, wherein the stressor is culturing the algae at a concentration where one or more nutrients are limiting.

11. The method of claim 1, wherein the algae comprise freshwater species, marine species, briny species of microalgae, species of microalgae that live in brackish water, or a combination of any two or more of the foregoing.

12. The method of claim 1, wherein the plurality of fish comprise freshwater species, marine species, briny species, species that live in brackish water, or a combination of any two or more of the foregoing.

13. The method of claim 1, wherein the algae composition comprises microalgae of at least one species of Cyanobacteria, Amphiprora, Chaetoceros, Isochrysis, Scenedesmus, Chlorella, Spirulena, Coelastrum, Micractinium, Euglena, or Dunaliella.

14. The method of claim 1, wherein the plurality of fishes comprise piscivores, herbivores, zooplanktivores, detritivores, or a combination of any two or more of the foregoing.

15. The method of claim 1, further comprising increasing or decreasing the total number of fish or the number of fish of any one or more species, in the plurality of fishes.

16. The method of claim 8, wherein the algae are concentrated by at least one round of foam fractionation.

17. The method of claim 1, wherein the aquatic composition is supplemented with carbon dioxide.

18. The method of claim 1, wherein the aquatic composition is supplemented with fly ashes.

19. The method of claim 2, wherein the system comprises open ponds on coastal land, and wherein the algae and the plurality of fish are marine species.

20. A method for making a liquid fuel comprising processing the biofuel feedstock produced by the method of claim 1.

21. The method of claim 20, wherein the liquid fuel is diesel, biodiesel, kerosene, jet-fuel, gasoline, JP-1, JP-4, JP-5, JP-6, JP-7, JP-8, Jet Propellant Thermally Stable (JPTS), a product of the Fischer-Tropsch process, an alcohol-based fuel, an ethanol-containing transportation fuel, algae cellulosic biomass-based liquid fuel, or pyrolysis oil-derived fuel.

22. A system for culturing algae comprising a growth enclosure comprising an aquatic composition in which algae and a plurality of fish are cultured, wherein one or more conditions of the aquatic composition are controllably modified by the plurality of fish to promote algal growth, an induction enclosure wherein the algae is induced to accumulate lipids by a stressor, a means for concentrating the algae, a means for harvesting the algae, and a means for extracting the lipids from the algae.

23. The system of claim 22, wherein said means for concentrating the algae comprises a foam fractionation unit.

24. The system of claim 22, wherein the growth enclosure is an open pond situated on coastal land, and the algae and the plurality of fish are marine species.
